(19) United States
(12) Patent Application Publication
(10) Pub. No.: US 2026/0232264 A1
(43) Pub. Date: Aug. 13, 2026

(54) Modular Garment Segment Pressurization Response

(71) Applicant: Vital Guard LLC, Belton, TX (US)

(72) Inventors: Alexander Joseph Robbins, Belton, TX (US); Aliasgar Morbi, USA, CA (US); Rohan Thakar, Ottawa (CA); Mathieu David Rancourt, Ottawa (CA)

(73) Assignee: Vital Guard LLC, Belton, TX (US)

(21) Appl. No.: 19/531,761

(22) Filed: Feb. 6, 2026

Related U.S. Application Data
(60) Provisional application No. 63/755,614, filed on Feb. 7, 2025.
(60) Provisional application No. 63/868,330, filed on Aug. 21, 2025.

(51) Int. Cl. A61B 5/00; A41D 13/05; A61B 5/0205; A61B 5/024

(52) U.S. Cl. CPC A61B 5/6805; A41D 13/0518; A61B 5/02055; A61B 5/7282; A61B 5/746; A61B 5/02416

(57) ABSTRACT

Apparatus and related methods generally relate to a wearable injury threat event detection apparatus. The wearable apparatus may, for example, be implemented as garment comprising at least one garment detection zone (GDZ) having a vacuum-sealed interior region. The interior region may include sensor module(s) configured to detect a change in pressure within the interior region. Depressurization of the interior region may, for example, trigger an alert, such as on a remote alert device (RAD) communicably coupled to the sensor module of the GDZ. Various embodiments may advantageously provide sensitive and cost-effective wearable injury threat detection, such as of penetration of a wearer's body.

### CROSS-REFERENCE TO RELATED APPLICATIONS

This application claims the benefit of U.S. Provisional Application No. 63/868,330, filed Aug. 21, 2025, and claims the benefit of U.S. Provisional Application No. 63/755,614, filed Feb. 7, 2025, both of which applications are titled “Modular Garment Segment Pressurization Response,” and name Alexander Joseph Robbins, et al, as inventors. The entire contents of each of the foregoing applications are incorporated herein by reference.

Unless expressly stated, changes in terminology from priority applications to this application are made without prejudice or disclaimer of subject matter. Changes from the priority application(s) (e.g., provisional applications(s)) are intended to be broadening and/or additive unless expressly stated otherwise. Replacement of alternative terms with a single representative term, for example, are inclusive unless otherwise defined. Various embodiments may also be found in previous disclosure(s) incorporated by reference. Embodiments of similar languages in this application are not modifications or disclaimer of the embodiments disclosed in previous incorporated disclosures unless otherwise stated.

### BACKGROUND

Law enforcement officers, military personnel, and first responders may, for example, wear specialized garments and/or equipment, which may be designed to enhance their performance, protection, and/or operational efficiency. Law enforcement officers may, for example, wear a duty uniform made from durable and/or breathable fabrics. This uniform may, for example, include reinforced stitching and multiple pockets. For example, the uniform may be configured to carry tools such as, by way of example and not limitation, handcuffs, a flashlight, a weapon, and/or other tools. Officers may, for example, wear body armor and/or ballistic vests, which may be configured, for example, to provide protection against firearms and/or other weapons. Their gear may, for example, include a duty belt. The duty belt may, for example, be configured to carry a firearm, radio, baton, pepper spray, and/or other tools for the officer's duties.

Military personnel's uniforms and equipment may, for example, be designed for various environments and/or missions. Their attire may, for example, include camouflage uniforms configured to blend into different terrains, which may, for example, range from woodland to desert patterns. These uniforms may be made, for example, from rugged materials configured to withstand harsh conditions. The uniforms may, for example, include features like moisture-wicking and/or flame-resistant properties. Military personnel may, for example, wear body armor and/or helmets configured, for example, to protect against ballistic threats (e.g., bullets, shrapnel). Their equipment may include a wide array of items such as, by way of example and not limitation: firearms, communication devices, night-vision goggles, tactical gloves, and/or combat boots.

Other first responders, such as firefighters and emergency medical technicians (EMTs) or paramedics, may, for example, have specialized garments and/or equipment tailored to their specific roles. Firefighters, for example, may wear turnout gear (e.g., also known as bunker gear), which may include, for example, a flame-resistant coat, pants, gloves, boots, and/or a helmet (e.g., with a visor). This gear may, for example, be designed to protect against heat, flames, and/or hazardous chemicals. Firefighters may, for example, carry rescue operations tools including, by way of example and not limitation, axes, hoses, and/or breathing apparatus. Medical responders (e.g., EMTs, paramedics) may, for example, wear uniforms that are easy to move in and/or equipped with reflective strips (e.g., for visibility). Medical responders may, for example, carry medical kits, communication devices, and/or personal protective equipment (PPE). PPE may, for example, include gloves and/or masks configured, for example, to allow them to provide medical care safely and/or efficiently.

### TECHNICAL FIELD

Apparatus and methods generally relate to response to potential injury of living beings.

### BRIEF SUMMARY

Apparatus and related methods generally relate to a wearable injury threat event detection apparatus. The wearable apparatus may, for example, be implemented as garment comprising at least one garment detection zone (GDZ) having a vacuum-sealed interior region. The interior region may include sensor module(s) configured to detect a change in pressure within the interior region. Depressurization of the interior region may, for example, trigger an alert, such as on a remote alert device (RAD) communicably coupled to the sensor module of the GDZ. Various embodiments may advantageously provide sensitive and cost-effective wearable injury threat detection, such as of penetration of a wearer's body.

### BRIEF DESCRIPTION OF THE DRAWINGS

Various embodiments of the present embodiments are described with reference to the following FIGURES.

FIG. 1 depicts a wearable automatic response module (WARM) coupled to a responsive apportioned garment (RAP) in an illustrative use-case scenario automatically responding to a detected injury threat event (DIRE).

FIG. 2A depicts a block diagram of an illustrative WARM communicably coupled to a remote alert device (RAD), such as depicted in FIG. 1.

FIG. 2B depicts a block diagram of a GDZ coupled to a RAD (e.g., directly).

FIG. 3 depicts an illustrative WARM configuration method.

FIG. 4 depicts an illustrative WARM operation method related to detecting and/or responding to a DIRE.

FIG. 5 depicts an illustrative block diagram depicting operation and/or training of an example DIRE detection engine.

FIG. 6 depicts an example garment detection zone (GDZ) of a RAP in an embodiment including an internal pressure detection sensor.

FIG. 7 depicts an example GDZ of a RAP in an embodiment including an expanding media and an electric attribute detection module.

FIG. 8 depicts an example GDZ of a RAP in an embodiment including a piezoelectric film.

FIG. 9 depicts an illustrative embodiment including an upper RAP and a lower RAP.

FIG. 10 and FIG. 11 depict an example releasably coupled WARM.

FIG. 12 depicts an example WARM, such as depicted in FIGS. 10-11, and a corresponding coupling module in an example garment (e.g., RAP).

FIG. 13 depicts an example multi-WARM docking station.

FIG. 14 depicts an illustrative user interface (UI) of an example RAD.

FIG. 15, FIG. 16A, and FIG. 16B depict illustrative RAPs in a vest format.

Appendix A depicts an example embodiment(s) of an example RAP including, by way of example and not limitation, wirelessly communicable GDZs.

Like reference numerals refer to like parts throughout the various views unless otherwise specified. Embodiments and portions of embodiments illustrated and described herein are non-limiting and non-exhaustive.

### DETAILED DESCRIPTION OF THE DRAWINGS

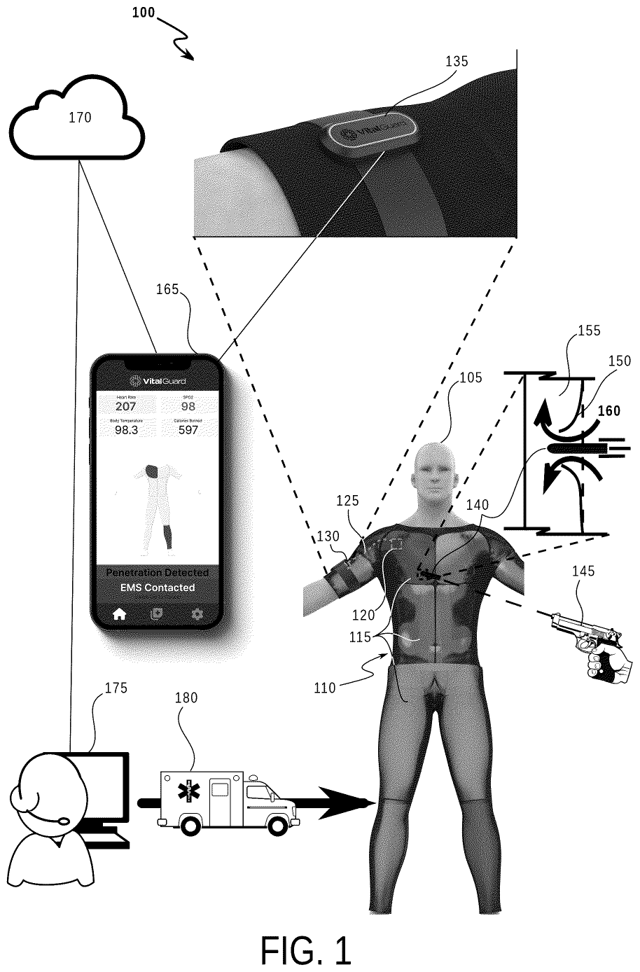

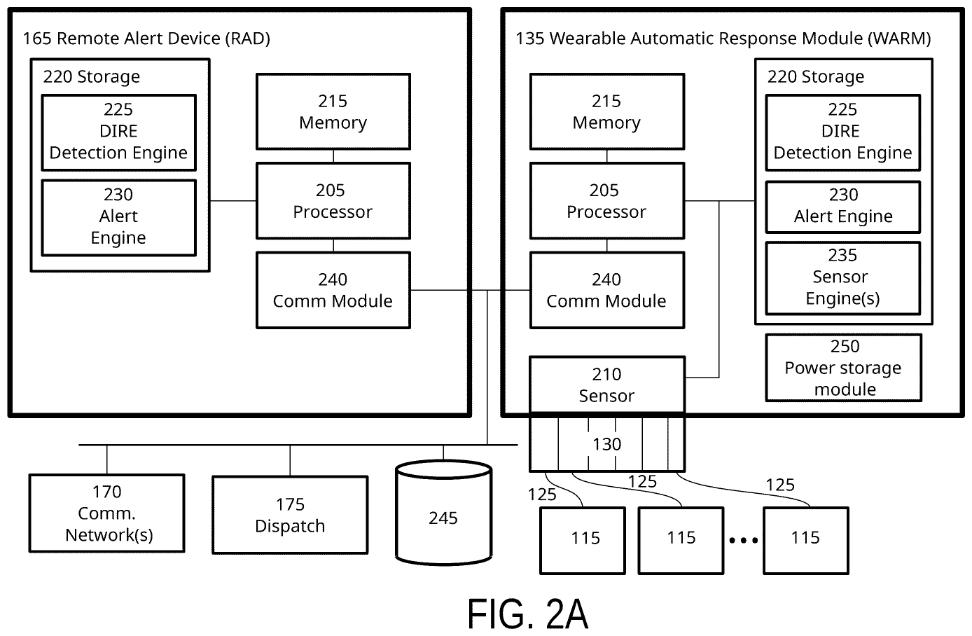

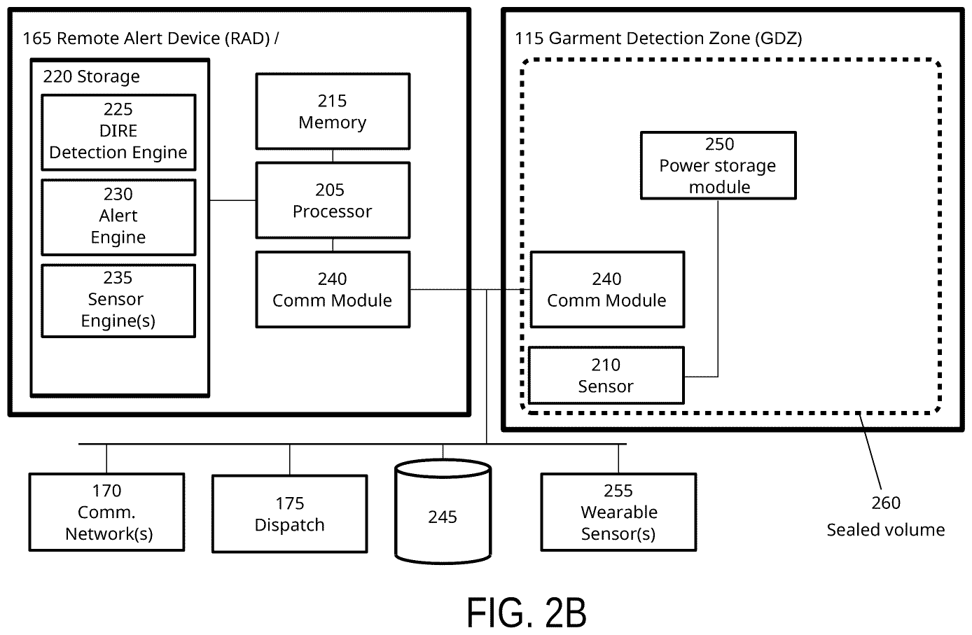

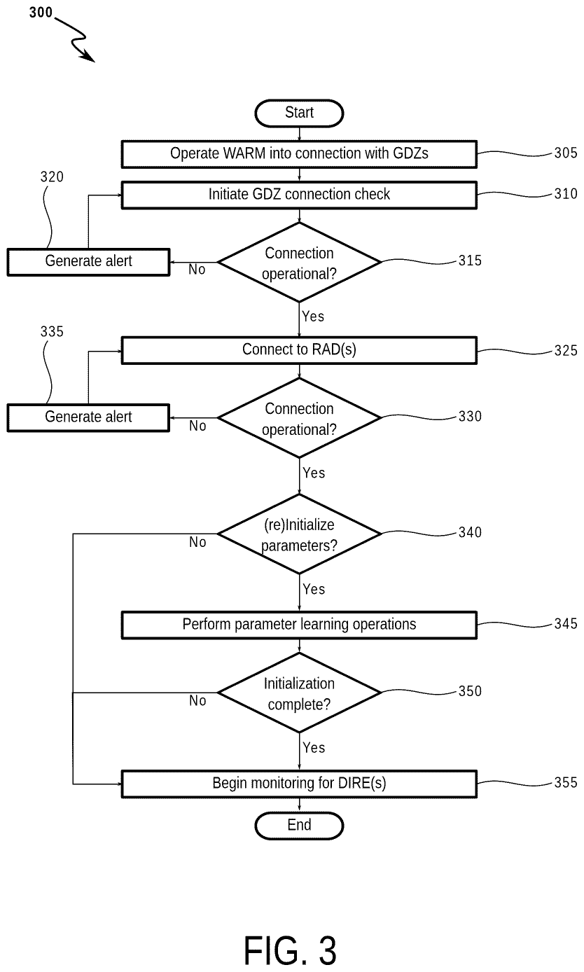

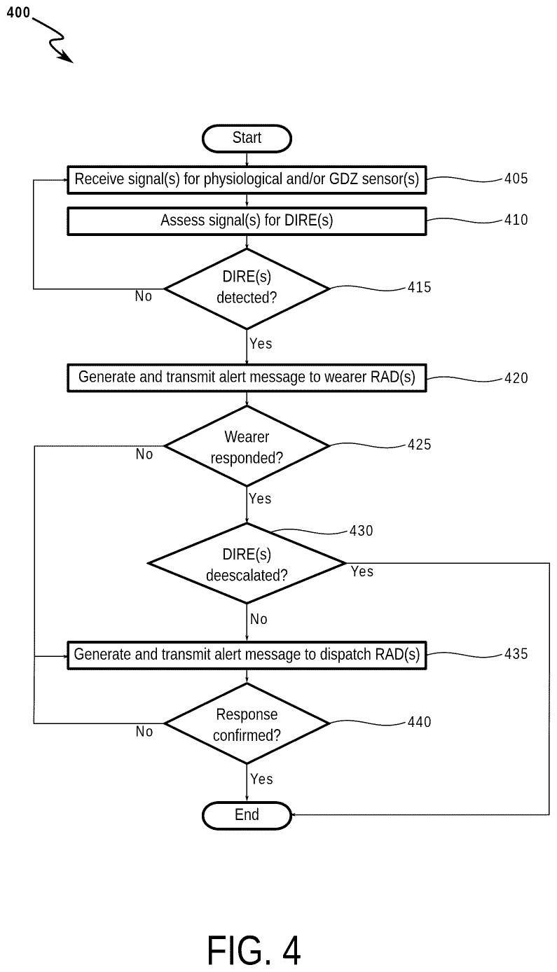

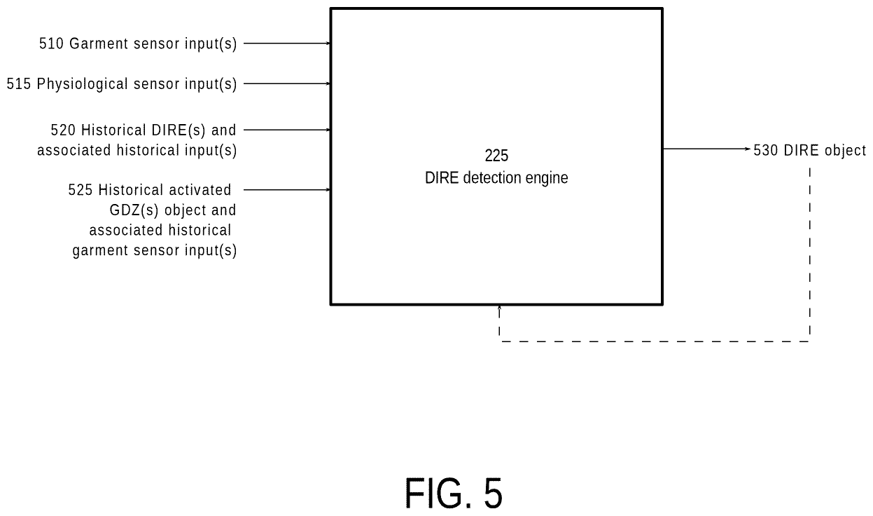

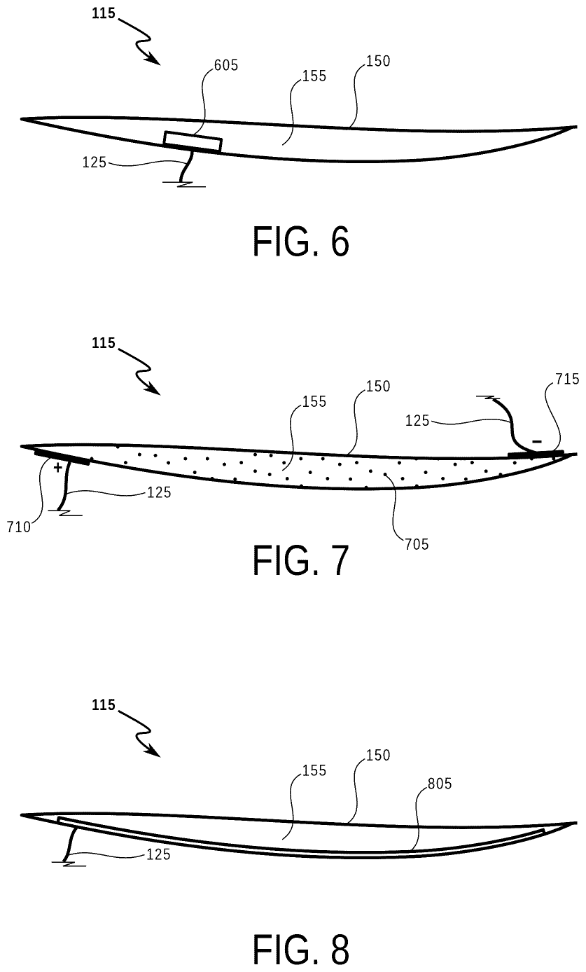

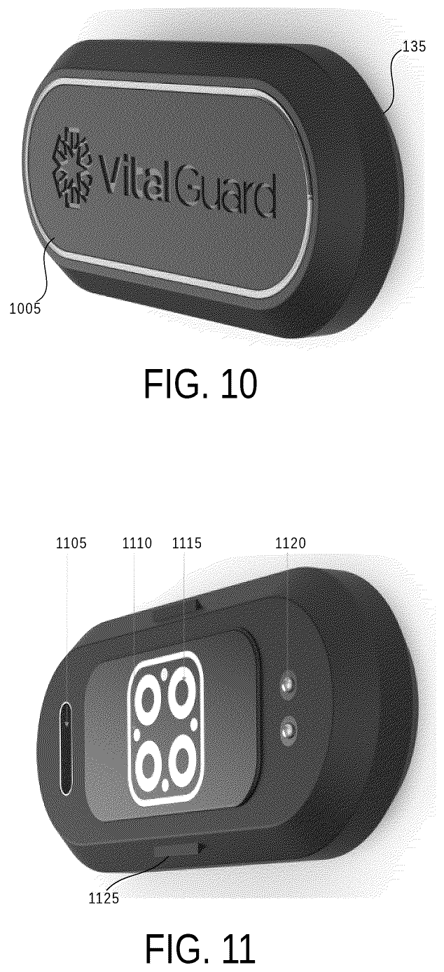

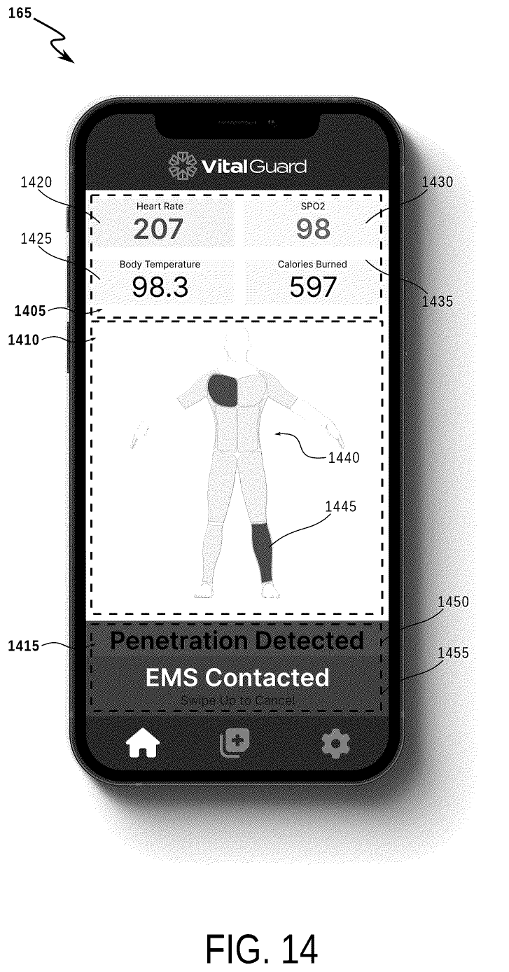

In order to assist rapid comprehension, this document introduces responsive apportioned garment (RAP) equipped with a wearable automatic response module (WARM) in FIGS. 1-2. Methods related to configuration and/or operation of a WARM are described with respect to FIGS. 3-4. The discussion turns to training of a detected injury threat event (DIRE) detection engine with reference to FIG. 5, such as may be embodied in a WARM, for example. Then, example RAP configurations are disclosed with respect to FIGS. 6-9. Example WARM embodiments and related charger(s) are then disclosed regarding FIGS. 10-13. Then, with respect to FIG. 14, this document discusses example graphical user interface(s) (GUI(s)) configured to interface with one or more WARM(s), for example. Finally, various additional embodiments and/or features are discussed related to WARM(s) and/or RAP(s).

FIG. 1 depicts a WARM coupled to a RAP in an illustrative use-case scenario automatically responding to a DIRE. In this example, a user 105 is wearing a RAP 110. The RAP(s) 110, in the depicted example, includes an upper RAP (e.g., configured as a shirt as shown) and a lower RAP (e.g., configured as pants as shown). In this example, the RAP(s) 110 is shown configured as a base garment (e.g., undergarment). In some embodiments, the RAP(s) 110 may be configured as an outer garment.

The RAP(s) 110 is provided with multiple garment detection zones (GDZs 115). For example, a GDZ 115 may be configured as a discrete detection region. For example, a GDZ(s) 115 may be configured to localize detection and/or response to a DIRE (e.g., injury). As an illustrative example, a (e.g., each) depicted GDZ(s) 115 includes a sensor module 120 (e.g., one or more). The sensor module(s) 120 is coupled by a communication channel 125. For example, the communication channel(s) 125 may include a communication and/or power link (e.g., wire, cable, communication bus, distributed communication link(s)). The communication channel(s) 125 communicably couples the sensor module(s) 120 to a coupling module 130. The coupling module(s) 130 couples (e.g., communication and/or power) the sensor module(s) 120 of the corresponding GDZ(s) 115 to a WARM 135. For example, the WARM(s) 135 may be releasably coupled to the RAP(s) 110. The coupling module(s) 130 may, for example, include a coupling mechanism configured to releasably couple the WARM(s) 135 (e.g., a snap mechanism, a magnetic mechanism, a latch mechanism).

The user 105 may, for example, be a first responder and/or military personnel. For example, the user 105 may be a law enforcement officer. In the depicted scenario, the user 105 has been injured in the line of duty by a projectile 140. As shown, the projectile 140 is a bullet fired from a weapon weapon 145 (e.g., handgun as shown).

As depicted by the red color of the affected GDZ 115 (in this example, the upper right from the perspective of the user 105). In this example, a lower left leg GDZ(s) 115 is also depicted as having been affected (e.g., the user 105 may have struck his leg during an altercation, or already been shot in the leg). The GDZ(s) 115 may, for example, be constructed as a pressurized bladder. When the projectile 140 penetrates into the GDZ(s) 115, a boundary wall 150 of the GDZ(s) 115 is breached as the projectile 140 enters the interior region 155 of the GDZ(s) 115. The interior region(s) 155 may, for example, be under a sub-atmospheric pressure (e.g., a vacuum). As the projectile 140 penetrates the boundary wall(s) 150, exterior volume 160 (e.g., air) at an elevated pressure relative to the interior region(s) 155 (e.g., atmospheric pressure) enters the interior region(s) 155. The sensor module(s) 120 may, for example, detect the change in pressure (e.g., elevation of pressure to atmospheric pressure). When the sensor module(s) 120 detects the change, a signal may, for example, be generated. The signal may be transmitted (e.g., via the communication channel(s) 125 and coupling module(s) 130) to the WARM(s) 135. For example, the signal may correspond to a DIRE. The signal may, for example, identify the GDZ(s) activated. Accordingly, the WARM(s) 135 and GDZ(s) 115 may, for example, advantageously enable rapid injury localization and/or danger level identification.

Sub-atmospheric volumes configured to detect threat of bodily injury (e.g., penetration such as by a bullet, knife, debris) may, for example, advantageously provide a combination of highly sensitive detection with cost-effective manufacturing. For example, vacuum-sealed pouches may be manufactured economically at scale. Detection of loss of pressure (e.g., exposure of a sensor to air, change in pressure, change in gas composition, release of pressure applied by walls of a vacuum-sealed container) may, for example, advantageously provide a highly-sensitive injury event mechanism. For example, ‘negative’ (e.g., sub-atmospheric) pressure is known to be a relatively unstable state. Triggering an alert based on loss of a vacuum may, for example, advantageously have an increased reliability relative to standard ‘impulse’ pressure detection, which may be difficult to distinguish from normal use (e.g., bending over, leaning against an object). Furthermore, rupture of a sealed volume may, for example, cause loss of predetermined pressure. Rupture of a sealed volume may, for example, occur in non-impulse scenarios (e.g., dragging of the wearer). Accordingly, various embodiments may advantageously provide an increased sensitivity (e.g., especially in a binary detection mode—injury or no injury) with a relatively low computation and/or low cost manufacturing configuration.

In this example, the WARM(s) 135 is communicably coupled (e.g., wirelessly) to a remote alert device (RAD 165). A RAD(s) 165 may, for example, include a personal computing device, such as a smartphone (as shown). For example, the RAD(s) 165 may be coupled to the WARM(s) 135 via near field communication (e.g., BLUETOOTH). The RAD(s) 165 may, for example, receive the signal (e.g., corresponding to a DIRE). As depicted, the RAD(s) 165 may alert the user 105 that a DIRE has been detected. The RAD(s) 165 may, for example, determine location and/or danger level (e.g., based on the signal and/or correlation to thresholds and/or historical events). Accordingly, the RAD(s) 165 may advantageously identify and/or communicate a DIRE and corresponding injury localization and/or danger level.

The RAD(s) 165 is communicably coupled to one or more communication networks 170. For example, a communication network 170 may include a cloud network. A communication network(s) 170 may, for example, include a navigational and/or positioning system (e.g., global positioning satellite (GPS) system). For example, the RAD(s) 165 may be communicably coupled to a dispatch system 175 (e.g., through the communication network(s) 170). In response to the DIRE, the RAD(s) 165 may generate and transmit to the dispatch system(s) 175 a signal alerting the dispatch system(s) 175 to the DIRE. In the depicted example, the dispatch system(s) 175 dispatches, in response to the DIRE, one or more responders 180. The responder(s) 180 may, by way of example and not limitation, include emergency medical services (e.g., an ambulance) and/or backup law enforcement. Accordingly, various embodiments may advantageously enable rapid automatic response (e.g., locally, remotely) to a detected injury threat event (DIRE) experienced by a wearer of the WARM.

FIG. 2A depicts a block diagram of an illustrative WARM communicably coupled to a remote alert device (RAD), such as depicted in FIG. 1. For example, the WARM(s) 135 may be the WARM(s) 135 of FIG. 1. As depicted, the WARM(s) 135 includes a sensor interface module 210. In the depicted example, the sensor interface module(s) 210 is communicably interfaced with the coupling module(s) 130 (e.g., of a RAP). The coupling module(s) 130 is coupled by corresponding communication channel(s) 125 to corresponding GDZ(s) 115. Accordingly, the sensor interface module(s) 210 may, as depicted, operably couple the WARM(s) 135 to the various communication channel(s) 125 of a RAP(s) 110.

In some implementations, the sensor interface module(s) 210 may, for example, include one or more sensors. For example, the sensor interface module(s) 210 may sense physical changes in the coupling module(s) 130. As an illustrative example, the communication channel(s) 125 may be configured as a distributed path (e.g., conductive textile) from the corresponding sensor module(s) 120. The coupling module(s) 130 may, for example, be a physical connector. For example, the sensor interface module(s) 210 sensor(s) may detect a raw signal from the coupling module(s) 130. The sensor interface module(s) 210 sensor(s) may, for example, detect capacitance. For example, a change in a GDZ (e.g., vacuum release, penetration) may change a capacitance. In some embodiments, the sensor interface module(s) 210 may, for example, detect resistance. A change in a GDZ may, for example, change resistance. In some embodiments, the sensor interface module(s) 210 may, for example, detect inductance. A change in a GDZ (e.g., penetration, blunt force) may, for example, change resistance.

In this example, the WARM(s) 135 includes a processor 205. A processor(s) 205 may, by way of example and not limitation, include multiple processors. The processor(s) 205 may include, for example, one or more microprocessors. The processor(s) 205 is operably coupled to memory 215. The memory 215 may, for example, include one or more physical modules. For example, the memory 215 may include random access memory. The memory 215 may be configured, by way of example and not limitation, to hold program(s) of instruction and/or operating data during and/or around execution by the processor(s) 205.

The processor(s) 205 is operably coupled to a storage module 220. The storage module(s) 220 may, for example, include multiple storage devices. For example, the storage module(s) 220 may include hard disk storage. The storage module(s) 220 may, for example, include solid state storage. The storage module(s) 220 may, for example, be configured to store one or more programs of instruction. A program of instruction may, for example, be configured to cause operations to be performed when executed by the processor(s) 205. For example, program(s) of instruction may be loaded (e.g., temporarily) from the storage module(s) 220 into the memory 215 such as, for example, in preparation for and/or during execution by the processor(s) 205.

As shown, in this example the storage module(s) 220 includes a DIRE detection engine 225. The DIRE detection engine(s) 225 may, for example, be configured to cause operations to be performed to generate a DIRE package (e.g., data object). For example, the DIRE package(s) may be generated in response to incoming signals (e.g., from the sensor interface module(s) 210) and/or historical data. For example, the DIRE detection engine(s) 225 may operate on signal(s) from the sensor interface module(s) 210. The DIRE package may, for example, include an indication of a DIRE. The DIRE package may include, for example, a corresponding time (e.g., of detection time of the DIRE, a current duration of the DIRE). The DIRE package may include, for example, a type(s) of DIRE (e.g., penetration, blunt trauma, weather, physiological parameters, non-communication). The DIRE package may include, for example, a location(s) corresponding to the DIRE (e.g., GDZ(s), location on the body, navigational positioning such as, for example, GPS coordinates).

The storage module(s) 220 may include, as depicted, an alert engine 230. The alert engine(s) 230 may, for example, be configured to cause operations to be performed to generate an alert package. For example, the alert package may be generated based on one or more DIRE packages. The alert package may, for example, be configured to induce a device to perform alert operations when the alert package is transmitted to the device. The device may include, for example, a RAD (e.g., RAD(s) 165).

In some embodiments, the alert engine(s) 230 may receive an alert package (e.g., a previously generated alert package, an alert package from another alert engine(s) 230). The alert engine(s) 230 may perform alert operations in response to receiving an alert package, for example.

Alert operations may, for example, include generating graphical user interface (GUI) alerts. The GUI alerts may be interactive, for example. The alert operations may, for example, include generating audible alerts. The alert operations may, for example, include generating tactile (e.g., haptic such as vibration) alerts. The alert operations may, for example, include generating thermal alerts. The alert operations may, for example, include generating dispatch instructions. The alert operations may, for example, be directed towards a user (e.g., RAP wearer). The alert operations may, for example, be directed to management personnel. The alert operations may, for example, be directed to emergency responders. The alert operations may, for example, be directed to devices (e.g., dispatch devices, RADs). An alert package may, for example, define one or more alert operations and/or parameters corresponding to alert operations.

The storage module(s) 220 may include, as depicted, one or more sensor engine 235. A sensor engine(s) 235 may, for example, be configured to cause sensing operations to be performed. The sensor engine(s) 235 may, for example, receive signals from a sensor(s) (e.g., sensor module(s) 120 via communication channel(s) 125 and sensor interface module(s) 210). The sensor engine(s) 235 may, for example, generate conditioned signal(s) and/or data objects from the sensor signal(s). As an illustrative example, the sensor engine(s) 235 may receive a signal from a pressure sensor (e.g., sensor module(s) 120). The sensor engine(s) 235 may, for example, interpret the signal from the pressure sensor to determine a pressure level. The sensor engine(s) 235 may, for example, determine whether a change in pressure has occurred. The sensor engine(s) 235 may, for example, generate an output (e.g., signal, data object). The DIRE detection engine(s) 225 may, in some embodiments (e.g., for one or more sensors), receive sensor data from the alert engine(s) 230.

In some embodiments, the sensor engine(s) 235 may, for example, process signals from distributed communication paths into a physical measurement. As an illustrative example, sensor module(s) 120 may be communicably coupled (e.g., electrically coupled) to one or more distributed communication links. For example, the communication channel(s) 125 may include a conductive textile. For example, a RAP(s) 110 may include conductive fabric. The sensor interface module(s) 210 may, for example, detect changes in a physical parameter (e.g., an electrical attribute such as conductivity, resistance, capacitance, inductance, voltage) corresponding to manipulation of a GDZ. For example, penetration of a first GDZ may cause a first change in resistance. Penetration of a second GDZ may, for example, cause a second change in resistance. The first and second changes may be different. For example, even if each GDZ is not specifically indexed (e.g., the sensor interface module(s) 210 may receive signals corresponding to multiple different GDZs without a corresponding unique identification of which GDZ(s) originated the signal), each GDZ and/or an event (e.g., penetration) associated with a particular GDZ may correspond, by way of example and not limitation, to a unique ‘signature.’ Accordingly, the sensor interface module(s) 210 (e.g., after processing by a sensor engine(s) 235) may, for example, advantageously identify which GDZ(s) corresponds to a DIRE, even in embodiments having non-unique communication links (e.g., distributed communication channel(s) 125 such as a conductive garment).

The WARM(s) 135 includes, in this example, a communication module 240. The communication module(s) 240 may, for example, include a wireless communication module. The wireless communication module may, for example, include a transmitter (e.g., radio) and/or receiver. The communication module(s) 240 may, for example, include a wired communication module (e.g., Ethernet port).

In this example, the WARM(s) 135 is communicably coupled to a RAD(s) 165, such as by corresponding communication module(s) 240, as shown. The RAD(s) 165, in the depicted example, includes a processor(s) 205. The processor(s) 205 is operably coupled to a memory 215. The processor(s) 205 is operably coupled to a storage module(s) 220. As depicted, the storage module(s) 220 includes a DIRE detection engine(s) 225. The storage module(s) 220 includes, in this example, an alert engine(s) 230.

In some embodiments, the DIRE detection engine(s) 225 and/or the alert engine(s) 230 may, for example, operate as disclosed at least with reference to the WARM(s) 135. In some embodiments, the WARM(s) 135 or the RAD(s) 165 may omit a DIRE detection engine(s) 225. The WARM(s) 135 or the RAD(s) 165 may, in some embodiments, omit an alert engine(s) 230.

In some embodiments, for example, the WARM(s) 135 may include a DIRE detection engine(s) 225. The WARM(s) 135 may transmit a generated DIRE package(s) to the RAD(s) 165. The RAD(s) 165 may include an alert engine(s) 230 configured to operate on the received DIRE package(s). In some embodiments, the RAD(s) 165 may include, for example, a DIRE detection engine(s) 225 configured to receive a DIRE package(s) and generate an updated DIRE package(s) (e.g., by adding additional information available to the RAD(s) 165). Such embodiments may, by way of example and not limitation, advantageously provide reduced cost and/or power for a WARM(s) 135.

In some embodiments, the WARM(s) 135 may transmit outputs from one or more sensor engine(s) 235 directly to the RAD(s) 165. The RAD(s) 165 may, for example, include a DIRE detection engine(s) 225 configured to operate directly on the received outputs. Such embodiments may, by way of example and not limitation, advantageously provide a low-cost, low-power, and/or compact WARM(s) 135.

In some implementations, the WARM(s) 135 may include a DIRE detection engine(s) 225, as depicted. The DIRE detection engine(s) 225 may, for example, generate a DIRE package(s). The WARM(s) 135 may transmit the DIRE package(s) to the RAD(s) 165. The RAD(s) 165 may, by way of example and not limitation, contain an alert engine(s) 230. For example, the RAD(s) 165 may not include a DIRE detection engine(s) 225 or the DIRE detection engine(s) 225 may, for example, be disabled (e.g., temporarily, selectively). The alert engine(s) 230 of the RAD(s) 165 may, for example, operate directly on the received DIRE package(s). Such embodiments may, for example, advantageously reduce computational load on the RAD(s) 165.

In some implementations, the RAD(s) 165 may include an alert engine(s) 230 (as depicted). The WARM(s) 135 may include, for example, an alert engine(s) 230 (as depicted). The alert engine(s) 230 of the RAD(s) 165 may, for example, receive an alert package(s) generated by the alert engine(s) 230 of the WARM(s) 135. The alert engine(s) 230 of the RAD(s) 165 may, for example, induce alert operations on the RAD(s) 165 (e.g., GUI display operations, dispatch operations, communications operations, warning operations). Such embodiments may, for example, advantageously provide increased compatibility of the WARM(s) 135 with multiple device types (e.g., smartphones, tablets, internet of thing (IOT) devices), such as, for example, by reducing hardware and/or software on the RAD(s) 165.

In this example, the communication module(s) 240 of the RAD(s) 165 and/or WARM(s) 135 is communicably coupled to a communication network(s) 170. As an illustrative example, the communication module(s) 240 of the WARM(s) 135 may be communicably coupled to a geo-positioning navigational (e.g., satellite) system. The communication module(s) 240 of the WARM(s) 135 may, for example, be communicably coupled to a distributed communication network (e.g., cellular, satellite, wireless area network). The communication module(s) 240 of the RAD(s) 165 and/or WARM(s) 135 may, for example, be communicably coupled to a cloud communication network (e.g., an emergency response network). The RAD(s) 165 and/or the WARM(s) 135 may, by way of example and not limitation, transmit a DIRE package(s) and/or alert package via the communication network(s) 170.

In the depicted example, the communication module(s) 240 of the RAD(s) 165 and/or the WARM(s) 135 is communicably coupled to a dispatch system 175. For example, the dispatch system(s) 175 may be communicably coupled via one or more of the communication network(s) 170.

As depicted, the WARM(s) 135 and/or the RAD(s) 165 are operably coupled to a data store(s) 245. The data store(s) 245 may include, for example, a database. The data store(s) 245 may include, for example, a physical storage device. The data store(s) 245 may include, for example, a virtual storage network.

The data store(s) 245 may, for example, store parameters. Parameters may, for example, include DIRE definitions (e.g., for use by a DIRE detection engine(s) 225). Parameters may, for example, include sensor calibration profiles (e.g., for use by a sensor engine(s) 235). Parameters may, for example, include alert definitions, alert logic, and/or alert sequences (e.g., for use by an alert engine(s) 230).

In some embodiments, the data store(s) 245 may include, for example, historical data. The data store(s) 245 may include, for example, historical DIRE packages. The data store(s) 245 may include historical alerts, for example. The data store(s) 245 may, for example, include historical sensor readings and/or outputs (e.g., from sensor engine(s) 235).

The data store(s) 245 may, for example, include associations between corresponding historical data. For example, associations may correlate corresponding historical sensor data, DIRE package(s), alerts, user responses, and/or outcomes. An association may, for example, be in the form of a matrix entry. An association may, for example, include a link attribute (e.g., identifier) to a corresponding data object. Historical data and/or corresponding associations may, for example, advantageously provide training and/or contextual data to one or more engine(s) (e.g., in a storage module(s) 220).

In this example, the WARM(s) 135 includes a power storage module 250. The power storage module(s) 250 may, for example, be operably coupled (not specifically shown) to provide power to the processor(s) 205 and/or other components (e.g., directly, indirectly). The power storage module(s) 250 may, for example, include a battery. The power storage module(s) 250 may include, for example, a power port and/or associated circuitry. The power storage module(s) 250 may include, for example, a charge control and/or power supply circuit(s).

FIG. 2B depicts a block diagram of a GDZ coupled to a RAD (e.g., directly). In some embodiments, such as depicted, the system may operate without a WARM (e.g., WARM(s) 135). For example, the GDZ(s) 115 may be communicably coupled to the RAD(s) 165. The GDZ(s) may, for example, be (e.g., each) coupled directly to a RAD(s). Such embodiments may, for example, advantageously reduce system complexity and/or cost, such as by reducing intermediate communication module(s).

As depicted, the GDZ(s) 115 includes a sealed volume 260. The sealed volume(s) 260 may, for example, be configured as a vacuum-sealed interior region. The sealed volume(s) 260 may, for example, be maintained at sub-atmospheric pressure. The sealed volume(s) 260 may, for example, be enclosed by boundary wall(s) (e.g., boundary wall(s) 150).

In this example, the GDZ(s) 115 includes sensor(s) 255. The sensor(s) 255 may, for example, be disposed within the sealed volume(s) 260. The sensor(s) 255 may, for example, include a pressure sensor. The sensor(s) 255 may, for example, include a piezoelectric film. The sensor(s) 255 may, for example, include expandable media and an electric attribute detection module, such as disclosed at least with reference to FIGS. 6-8. The sensor(s) 255 may, for example, be configured to detect a change in pressure within the sealed volume(s) 260.

The GDZ(s) 115 includes, in the depicted example, a power storage module 250. The power storage module(s) 250 may, for example, include a battery. The battery may, for example, be configured to power the sensor(s) 255, the communication module 240, and/or other components of the GDZ(s) 115. In some embodiments, the battery may include a circular battery (e.g., coin cell). The battery may, for example, advantageously provide long battery life (e.g., 6 months or more). In some implementations, the power storage module(s) 250 may include a rechargeable battery. For example, the battery may be wirelessly rechargeable. A GDZ (e.g., in a garment, removed from a garment) may be subjected to a wireless charging field, such as to recharge the power storage module(s) 250. In some embodiments, for example, the GDZ may include a charging control circuit(s).

As shown, the GDZ(s) 115 includes a communication module 240. The communication module(s) 240 may, for example, include a wireless communication module. The wireless communication module may, for example, include a Bluetooth module. The Bluetooth module may, for example, be configured to transmit data wirelessly to the RAD(s) 165. In some embodiments, the communication module(s) 240 may utilize coded Bluetooth signals. Coded Bluetooth signals may, for example, advantageously achieve secure communication. The wireless design may, for example, advantageously facilitate maintaining vacuum seal integrity of the sealed volume(s) 260. For example, wireless communication may advantageously avoid wired connections that could compromise the vacuum seal. Wireless communication may, for example, advantageously permit an entirely sealed volume 260, such as without any physical elements (e.g., data conduits such as wires/cables) traversing the boundary of the sealed volume. Although shown in the context of the depicted embodiments, GDZs of various embodiments (e.g., of FIG. 2A) may, for example, be provided with a sealed volume 260.

In some embodiments, the GDZ(s) 115 may include a printed circuit board (PCB). The PCB may, for example, include the communication module(s) 240 and the sensor(s) 255. The PCB may, for example, include a controller communicably coupled to the sensor(s) 255. The controller may, for example, be configured to perform substantially continuous pressure monitoring (e.g., polled at <1 min frequency, sampled at 1 Hz frequency, sampled greater than 1 Hz). Continuous pressure monitoring may, for example, advantageously detect sudden changes in pressure.

In some implementations, the GDZ(s) 115 may include a housing. The housing may, for example, be configured to protect the electronics (e.g., PCB, sensor(s) 255, communication module(s) 240, power storage module(s) 250). The housing may, for example, be constructed from plastic. The plastic may, for example, be 3D printed, molded, and/or formed. The housing may, for example, offer flexibility in design and/or material selection.

The RAD(s) 165, in the depicted example, includes components as disclosed at least with reference to FIG. 2A. For example, the RAD(s) 165 includes a processor(s) 205, memory 215, and storage module(s) 220. The storage module(s) 220 includes, as shown, a DIRE detection engine(s) 225 and an alert engine(s) 230. The storage module(s) 220 may include, in some embodiments, one or more sensor engine(s) 235. The RAD(s) 165 includes a communication module(s) 240 configured to communicably couple to the GDZ(s) 115 (e.g., wirelessly via Bluetooth).

In this embodiment, the RAD(s) 165 may receive signals directly from the GDZ(s) 115. For example, the sensor(s) 255 may detect a change in pressure within the sealed volume(s) 260. The communication module(s) 240 of the GDZ(s) 115 may transmit a signal corresponding to the detected change to the RAD(s) 165. The DIRE detection engine(s) 225 of the RAD(s) 165 may, for example, operate on the received signal to detect a DIRE. The alert engine(s) 230 may, for example, generate an alert in response to the detected DIRE.

As depicted, the RAD(s) 165 is communicably coupled to one or more communication network(s) 170. The RAD(s) 165 may, for example, transmit alerts and/or DIRE packages via the communication network(s) 170 to a dispatch system(s) 175. The RAD(s) 165 is operably coupled, in this example, to a data store(s) 245, such as disclosed at least with reference to FIG. 2A.

In the depicted embodiment, the system includes a wearable sensor(s) 255. The wearable sensor(s) 255 may, for example, include a consumer wearable device. The wearable sensor(s) 255 may, for example, be embodied in a fitness tracker. The wearable sensor(s) 255 may, for example, be embodied in a smartwatch. The wearable sensor(s) 255 may, for example, be configured to collect physiological data.

The wearable sensor(s) 255 may, for example, be communicably coupled to the RAD(s) 165 (as shown) and/or the GDZ(s) 115. The communication may, for example, be wireless (e.g., via Bluetooth, Wi-Fi). The wearable sensor(s) 255 may, for example, transmit physiological sensor input(s) (e.g., physiological sensor input(s) 515) to the RAD(s) 165. The physiological sensor input(s) may include, by way of example and not limitation, heart rate data, blood oxygen data, temperature data, activity data, and/or calorie expenditure data.

In some implementations, the DIRE detection engine(s) 225 may, for example, receive physiological sensor input(s) from the wearable sensor(s) 255. The DIRE detection engine(s) 225 may, for example, fuse the physiological sensor input(s) with signals from the GDZ(s) 115 to detect and/or classify a DIRE. For example, the DIRE detection engine(s) 225 may correlate a detected pressure change in a GDZ(s) 115 with a sudden change in heart rate detected by the wearable sensor(s) 255. Such correlation may, for example, advantageously enhance accuracy of DIRE detection and/or classification.

The integration of the wearable sensor(s) 255 may, for example, advantageously avoid a dedicated vital-sign module embedded in the garment. Such embodiments may, for example, reduce hardware cost and/or complexity. The wearable sensor(s) 255 may, for example, advantageously leverage existing consumer devices that a user (e.g., user 105) may already own and/or wear.

In some embodiments, multiple GDZ(s) 115 may be communicably coupled to the RAD(s) 165. Each GDZ(s) 115 may, for example, operate as a standalone unit. Each GDZ(s) 115 may, for example, independently communicate with the RAD(s) 165. For example, a first GDZ(s) 115 may be positioned at a front portion of a garment, and a second GDZ(s) 115 may be positioned at a rear portion of the garment. Each GDZ(s) 115 may, for example, include its own sensor(s) 255, communication module(s) 240, and power storage module(s) 250. The RAD(s) 165 may, for example, receive signals from multiple GDZ(s) 115 and identify which GDZ(s) 115 has detected a pressure change. Such embodiments may, for example, advantageously provide injury localization.

The wireless configuration depicted in FIG. 2B may, for example, advantageously reduce physical connections between components. Reducing physical connections may, for example, enhance the product's flexibility. The configuration may, for example, facilitate ease of use. The configuration may, for example, increase the longevity of the vacuum seal in the sealed volume(s) 260. The configuration may, for example, enhance the reliability of the vacuum seal. Manufacturing costs of the vacuum-sealed GDZ(s) 115 may, for example, be reduced by removing wired connections that would otherwise penetrate the sealed volume(s) 260.

FIG. 3 depicts an illustrative WARM configuration method 300. The method 300, in the depicted example, begins with a step 305 of operating the WARM (e.g., WARM(s) 135) into connection with one or more GDZs. For example, the step 305 may include coupling the WARM to a RAP (e.g., into connection with a coupling module(s) 130). The step 305 may, for example, occur when a user 105 couples the WARM to their RAP (e.g., at the beginning of a shift).

In a step 310, a GDZ connection check is initiated. The step 310 may, for example, be triggered when the WARM is connected to the RAP and/or when the WARM is turned on. In some examples, the step 310 may be initiated when the WARM is operated into a connection check mode. In some examples, the step 310 may be initiated, for example, via a remote interface (e.g., via the RAD(s) 165).

The connection check may, for example, be automatic. For example, the connection check may inject a signal and/or monitor for a received signal (e.g., via the coupling module(s) 130). In some embodiments, the connection check may include, by way of example and not limitation, operations by a person (e.g., user 105). For example, the connection check may include prompting a user to manipulate one or more GDZs (e.g., in an instructed sequence).

If it is determined (e.g., by a sensor interface module(s) 210, sensor engine(s) 235, and/or DIRE detection engine(s) 225), in a decision point 315, that the connection is not operational, then an alert is generated (e.g., by an alert engine(s) 230) in a step 320, and the method 300 returns to the step 310. Once it is determined, at the decision point 315, that the connection is operational, then the method 300 proceeds to step 325.

In the step 325, the WARM connects to one or more RAD(s) (e.g., RAD(s) 165). For example, the step 325 may include communicating (e.g., directly, through a network) with a selected (e.g., user-selected, previously connected, default) RAD. In some embodiments, the step 325 may include, for example, connecting to a network (e.g., communication network(s) 170). For example, the step 325 may include prompting a user to select a communication mode and/or enter communication parameters. For example, the step 325 may include prompting a user to perform pairing operations and/or network connection operations.

If the connection is determined, in a decision point 330, to not be operational, an alert is generated (in this example) in a step 335. The alert may, for example, be generated by an alert engine(s) 230, and the method 300 returns to the step 325. Once it is determined, at the decision point 330, that the connection to the RAD(s) is operational, then the method 300 proceeds to a decision point 340.

If it is determined, at the decision point 340, that parameters are to be initialized (e.g., re-initialized), the method 300 proceeds to a step 345. Otherwise, the method 300 proceeds to a step 355. The decision point may, for example, include automatic determination (e.g., if this is a first use). The parameters may, for example, include DIRE detection and/or generation parameters. The parameters may, for example, include communication parameters. The parameters may, for example, include sensor parameters. The parameters may, for example, include alert parameters.

At the step 345, parameter learning operations are performed. In some embodiments, parameter learning operations may, for example, include loading predetermined parameters (e.g., from one or more data store(s) 245).

In some implementations, the parameter learning operations may, for example, include teaching operations. For example, the operations may include generating parameters based on corresponding inputs with associated outputs (e.g., historical, manually associated). The learning operations may, for example, include a training phase. The training phase may, for example, be conducted using retrieved historical data (e.g., from one or more data store(s) 245). The historical data may, for example, be divided into training and test data. The training data may be used, for example, to train one or more model(s) (e.g., of an engine such as a DIRE detection engine(s) 225, alert engine(s) 230, and/or sensor engine(s) 235).

In a test phase, for example, the model(s) may be applied to input data of the test data to generate output(s). The generated output(s) may, for example, be compared to the corresponding output data of the test data. An accuracy metric (e.g., difference) may be determined, for example. If the accuracy metric is within a training accuracy criterion(s) (e.g., a predetermined criterion(s), such as minimum accuracy threshold), then the step 345 may, for example, be determined (e.g., in a decision point 350) performed.

If the accuracy metric is not within the training accuracy criterion(s), the training operations may continue (e.g., on the same and/or further training data). In some implementations, training operations may be repeated (e.g., periodically, on demand, during operation, continuously).

If the initialization is determined, in the decision point 350, to not be performed, then the method 300 returns to the step 345. The decision point 350 may include, for example, a training test phase, such as disclosed above. In some examples, the decision point 350 may, for example, include completion of a predetermined sequence of initialization operations. In some embodiments, for example, the decision point 350 may, for example, include checking that certain parameters (e.g., core parameters, variables, all parameters) are performed.

Once it is determined, in the decision point 350, that initialization is performed, the WARM is operated into an operational mode in a step 355 (e.g., monitoring for DIRE(s), as depicted).

In some embodiments, the configuration method 300 may be adapted for systems operating without a WARM(s) 135. For example, in embodiments where GDZ(s) 115 communicate directly with a RAD(s) 165 (e.g., as disclosed at least with reference to FIG. 2B), the step 305 may be omitted or modified. In such embodiments, the method 300 may begin with a step of powering on the GDZ(s) 115. Powering on the GDZ(s) 115 may, for example, occur automatically when the GDZ(s) 115 is inserted into the garment and/or when the user 105 dons the garment. In some implementations, the GDZ(s) 115 may include a power switch and/or activation mechanism that enables the user 105 to manually power on the device.

In embodiments without a WARM(s) 135, the step 310 may include a GDZ self-test procedure. The self-test procedure may, for example, be performed by a controller (e.g., microcontroller, processor) disposed within the GDZ(s) 115. The self-test may, for example, include verifying the integrity of the vacuum seal by monitoring the pressure sensor(s) 255. The self-test may include, for example, verifying the functionality of the communication module(s) 240 by attempting to establish a wireless connection (e.g., Bluetooth connection) with the RAD(s) 165. The self-test may include, for example, checking the battery level of the power storage module(s) 250. If the self-test fails (e.g., vacuum seal compromised, communication module non-functional, battery depleted), the GDZ(s) 115 may generate an alert. The alert may, for example, include a visual indicator (e.g., LED light) on the GDZ(s) 115 itself and/or a signal transmitted to the RAD(s) 165.

The step 325 may, in embodiments without a WARM(s) 135, include establishing direct wireless communication between each GDZ(s) 115 and the RAD(s) 165. For example, each GDZ(s) 115 may independently pair with the RAD(s) 165 via Bluetooth. The pairing process may, for example, be automatic (e.g., using Bluetooth Low Energy auto-pairing). In some implementations, the pairing process may rely on user intervention. For example, the user 105 may be prompted by the RAD(s) 165 to confirm pairing with each detected GDZ(s) 115. The RAD(s) 165 may, for example, display a list of available GDZ(s) 115 and prompt the user 105 to select which GDZ(s) 115 to pair with. Such embodiments may, for example, advantageously prevent accidental pairing with GDZ(s) 115 belonging to other users in proximity.

In some embodiments, the step 325 may include assigning unique identifiers to each GDZ(s) 115. The unique identifiers may, for example, correspond to the physical location of each GDZ(s) 115 on the garment (e.g., “front torso,” “rear torso,” “left shoulder,” “right leg”). The assignment may, for example, be performed automatically based on pre-configured settings stored in the GDZ(s) 115 during manufacturing. In some implementations, the assignment may be performed manually by the user 105 via the RAD(s) 165. For example, the RAD(s) 165 may display a graphical representation of the garment (e.g., as shown in FIG. 14, GDZ display 1440) and prompt the user 105 to tap on the location corresponding to each GDZ(s) 115 as it is detected. Such embodiments may, for example, advantageously enable accurate injury localization even when GDZ(s) 115 are replaced or repositioned.

The decision point 340 and step 345 may, in embodiments without a WARM(s) 135, be performed by the RAD(s) 165 and/or by the individual GDZ(s) 115. For example, the RAD(s) 165 may include a DIRE detection engine(s) 225 that relies on initialization of DIRE detection parameters. The initialization may, for example, include establishing baseline pressure readings for each GDZ(s) 115. The baseline pressure readings may, for example, be obtained by querying each GDZ(s) 115 for its current pressure sensor reading while the vacuum seal is intact. The baseline readings may, for example, be stored in the data store(s) 245 and used as reference values for detecting pressure changes indicative of a DIRE.

In some implementations, the step 345 may include calibrating the sensor(s) 255 of each GDZ(s) 115. Calibration may, for example, involve adjusting sensor sensitivity, offset values, and/or gain parameters to account for manufacturing variations and/or environmental conditions. The calibration may, for example, be performed by the controller within each GDZ(s) 115 based on calibration algorithms stored in firmware. In some embodiments, the calibration may be performed by the RAD(s) 165 based on calibration data received from each GDZ(s) 115. The RAD(s) 165 may, for example, transmit calibration parameters back to each GDZ(s) 115 to update its sensor processing algorithms.

The step 345 may, in some embodiments, include configuring alert parameters. Alert parameters may, for example, include threshold values for pressure changes that trigger a DIRE detection. Alert parameters may include, for example, time windows for detecting simultaneous impacts across multiple GDZ(s) 115. Alert parameters may include, for example, escalation rules that define when and how alerts are transmitted to dispatch system(s) 175. The alert parameters may, for example, be configured based on user preferences, organizational policies, and/or historical data. In some implementations, the alert parameters may be remotely configured by a system administrator via the communication network(s) 170.

In embodiments without a WARM(s) 135, the step 355 may include activating continuous monitoring by each GDZ(s) 115. Continuous monitoring may, for example, involve the controller within each GDZ(s) 115 periodically sampling the sensor(s) 255 (e.g., at 1 Hz, at 10 Hz, at 100 Hz) and comparing the readings to baseline values. If a pressure change exceeding a predetermined threshold is detected, the GDZ(s) 115 may generate a signal and transmit it to the RAD(s) 165 via the communication module(s) 240. The RAD(s) 165 may, for example, receive the signal and invoke the DIRE detection engine(s) 225 to assess whether the signal corresponds to a DIRE (e.g., as disclosed at least with reference to FIG. 4).

In some embodiments, the configuration method 300 may include a step of establishing a self-assembling network among multiple GDZ(s) 115. For example, after each GDZ(s) 115 is powered on and paired with the RAD(s) 165, the GDZ(s) 115 may automatically discover other GDZ(s) 115 in proximity and establish peer-to-peer communication links. The self-assembling network may, for example, enable the GDZ(s) 115 to coordinate DIRE detection and/or share sensor data. The network formation may, for example, occur transparently to the user 105 without relying on manual configuration. The RAD(s) 165 may, for example, monitor the network topology and display the status of each GDZ(s) 115 (e.g., connected, disconnected, low battery) on a user interface (e.g., as shown in FIG. 14).

In some implementations, the configuration method 300 may include a step of synchronizing time across multiple GDZ(s) 115 and the RAD(s) 165. Time synchronization may, for example, enable accurate correlation of events detected by different GDZ(s) 115. For example, if multiple GDZ(s) 115 detect pressure changes within a short time window (e.g., within 100 milliseconds), the DIRE detection engine(s) 225 may determine that the events correspond to a single impact event rather than multiple independent events. Time synchronization may, for example, be achieved by the RAD(s) 165 broadcasting a reference time signal to all connected GDZ(s) 115. Each GDZ(s) 115 may, for example, adjust its internal clock to match the reference time.

The configuration method 300 may, in some embodiments, include a step of performing a functional test of the entire system. The functional test may, for example, involve the user 105 manually applying pressure to each GDZ(s) 115 in sequence while the RAD(s) 165 monitors for corresponding signals. The RAD(s) 165 may, for example, display visual feedback (e.g., highlighting the affected GDZ on the GDZ display 1440) to confirm that each GDZ(s) 115 is functioning correctly and that injury localization is accurate. If any GDZ(s) 115 fails to respond during the functional test, the RAD(s) 165 may generate an alert prompting the user 105 to check the GDZ(s) 115 (e.g., verify proper insertion, check battery level, verify vacuum seal integrity).

In embodiments where the system integrates with external wearable sensor(s) 255 (e.g., smartwatch), the configuration method 300 may include a step of pairing the wearable sensor(s) 255 with the RAD(s) 165. The pairing may, for example, occur via Bluetooth or another wireless protocol. The RAD(s) 165 may, for example, request permission from the wearable sensor(s) 255 to access physiological data (e.g., heart rate, blood oxygen saturation). Once permission is granted, the RAD(s) 165 may begin receiving physiological sensor input(s) 515 from the wearable sensor(s) 255. The DIRE detection engine(s) 225 may, for example, be configured to fuse the physiological sensor input(s) 515 with signals from the GDZ(s) 115 to increase DIRE detection accuracy.

The configuration method 300 may, in some embodiments, include a step of configuring communication with third-party emergency response APIs. The configuration may, for example, include authenticating the RAD(s) 165 with the API service, providing user profile information (e.g., name, badge number, organization), and/or configuring alert routing preferences. The configuration may, for example, be performed during initial setup and stored in the data store(s) 245 for subsequent use. When a DIRE is detected, the RAD(s) 165 may, for example, automatically transmit a DIRE package(s) 530 to the emergency response API, which may then route the alert to the appropriate dispatch system(s) 175.

In some implementations, the configuration method 300 may be performed partially or entirely by a remote configuration service. For example, a system administrator may remotely configure multiple RAD(s) 165 and associated GDZ(s) 115 via the communication network(s) 170. The remote configuration may, for example, include pushing firmware updates to the GDZ(s) 115, updating DIRE detection parameters, and/or modifying alert routing rules. Such embodiments may, for example, advantageously enable centralized management of large deployments (e.g., across an entire law enforcement agency) without relying on individual users to perform manual configuration.

FIG. 4 depicts an illustrative WARM operation method 400 related to detecting and/or responding to a DIRE. In this example, the method 400 begins in a step 405 when a signal(s) is received. As depicted, the signal(s) may, for example, correspond to a GDZ-associated sensor(s). The signal(s) may, for example, correspond to physiological sensor(s) (e.g., not associated with a particular GDZ(s)). The signal is assessed, in a step 410, for a DIRE(s) (e.g., by one or more DIRE detection engine(s) 225, such as after being processed by one or more sensor interface module(s) 210 and/or sensor engine(s) 235). If it is determined, in a decision point 415, that the signal(s) does not correspond to a DIRE, then the method 400 returns to the step 405. Otherwise, if a DIRE(s) is detected, then a signal is generated and transmitted, in a step 420, to the RAD(s) (e.g., RAD(s) 165) of the wearer (e.g., user 105). The signal may, for example, be an alert message as depicted. The signal may, for example, include a DIRE package(s) (e.g., generated by a DIRE detection engine(s) 225). The signal may, for example, include an alert package(s) (e.g., generated by an alert engine(s) 230).

The signal(s) generated and transmitted, in step 420, to the RAD(s) of the RAP's wearer, may, for example, be configured to induce the RAD(s) to generate an alert to the wearer. For example, the RAD(s) may notify the wearer that a DIRE(s) has been detected and prompt for a response. The requested response may, for example, be configured to be diagnostic to determine whether the DIRE(s) corresponds to a ‘false positive’ detection. The requested response(s) may, for example, be defined by the DIRE package(s) and/or alert package(s).

If it is determined, in a decision point 425, that the wearer has not responded, then the method 400 proceeds to a step 435. Otherwise, if it is determined that the wearer responded, then it is determined, in a decision point 430, whether the DIRE(s) has been deescalated. The WARM may, for example, determine that the wearer has responded based on a signal(s) received from the RAD(s). Deescalation may, for example, be determined based on a response received from the wearer.

As an illustrative example, the RAD may generate a notification (e.g., GUI and audible alert) indicating to the wearer that a DIRE has been detected. The notification may, for example, indicate the type of DIRE detected (e.g., hyperthermia, heat exhaustion, bodily penetration). The notification may, for example, prompt the wearer to indicate whether the DIRE is active. The notification may, for example, prompt the wearer to take steps to mitigate the DIRE (e.g., reducing activity and/or seeking shade for hyperthermia). The wearer's response may, for example, be monitored (e.g., by the WARM and/or RAD). If the wearer confirms mitigation actions have been taken, the DIRE may, for example, be deescalated (e.g., after confirmation that the triggering signal(s) are within normal limits). If the wearer confirms that the DIRE is not active (e.g., that no bodily penetration has occurred), the DIRE may, for example, be deescalated.

If it is determined, in the decision point 430, that the DIRE has been deescalated, then the method 400 ends. Otherwise, an alert signal(s) is generated and transmitted to one or more device(s). The alert signal(s) may, for example, be generated by an alert engine(s) 230. The alert may, for example, be generated and/or transmitted by the WARM. In some examples, the alert may, for example, be generated and/or transmitted by the RAD(s). The alert may, for example, be transmitted (e.g., via one or more communication network(s) 170) to a RAD(s). A RAD(s) may, for example, include a dispatch device (e.g., dispatch system(s) 175). The alert may, for example, be configured to induce dispatch of one or more responder(s) 180. The step 435 may, for example, be repeated until it is determined, in a decision point 440, that a response is confirmed. The response may, for example, be confirmed when an indication of a successful dispatch is received. The response may, for example, be confirmed when a user of a RAD (e.g., other than the wearer) confirms the alert has been received and/or acted upon. In some embodiments, repetition of the decision point 425 may result in increasing urgency and/or activation of backup measures (e.g., via additional or other communication methods and/or networks).

In some embodiments, the decision point 425, the decision point 430, the step 435, and/or the decision point 440 may, for example, be performed by the RAD(s). Such embodiments may, for example, advantageously reduce a computational load on the WARM(s) and/or reduce response time.

In some embodiments, one or more steps may be performed, for example, by a communicably coupled additional WARM(s). In some embodiments, one or more steps may, for example, be performed by a RAD(s). In some embodiments, one or more steps may be performed by a remote device (e.g., via a communication network(s) 170).

In some embodiments, the operation method 400 may be adapted for systems in other configurations, such as operating without a WARM(s) 135. For example, in embodiments where GDZ(s) 115 communicate directly with a RAD(s) 165 (e.g., as disclosed at least with reference to FIG. 2B), the method 400 may be performed primarily by the RAD(s) 165 and/or by distributed processing across multiple GDZ(s) 115. In such embodiments, the step 405 may include the RAD(s) 165 receiving signals directly from one or more GDZ(s) 115 via wireless communication (e.g., Bluetooth). Each GDZ(s) 115 may, for example, independently transmit signals corresponding to detected pressure changes within its sealed volume(s) 260.

In embodiments without a WARM(s) 135, the step 410 may be performed by the DIRE detection engine(s) 225 of the RAD(s) 165. The DIRE detection engine(s) 225 may, for example, receive signals from multiple GDZ(s) 115 in parallel and assess whether the signals correspond to a DIRE. The assessment may, for example, include comparing detected pressure changes to baseline pressure readings established during configuration (e.g., as disclosed at least with reference to FIG. 3). The assessment may include, for example, analyzing the temporal pattern of pressure changes to distinguish between true DIRE events and false positives (e.g., caused by normal movement, environmental pressure changes, accidental compression of the garment).

In some implementations, the step 410 may include fusing signals from multiple sources. For example, the DIRE detection engine(s) 225 may correlate signals from GDZ(s) 115 with physiological sensor input(s) 515 from external wearable sensor(s) 255 (e.g., smartwatch). If a GDZ(s) 115 reports a pressure change coincident with a sudden spike in heart rate and/or drop in blood oxygen saturation detected by the wearable sensor(s) 255, the DIRE detection engine(s) 225 may determine that a DIRE has occurred with high confidence. Conversely, if a GDZ(s) 115 reports a pressure change but physiological parameters remain normal, the DIRE detection engine(s) 225 may classify the event as a potential false positive for user confirmation.

In embodiments without a WARM(s) 135, the decision point 415 may be performed by the RAD(s) 165 based on the output of the DIRE detection engine(s) 225. If no DIRE is detected, the RAD(s) 165 may, for example, continue monitoring by returning to step 405. The RAD(s) 165 may, for example, maintain a log of all received signals for subsequent analysis and/or training of the DIRE detection engine(s) 225. In some implementations, the RAD(s) 165 may periodically query each GDZ(s) 115 for status updates (e.g., battery level, vacuum seal integrity, communication link quality) to ensure the system remains operational.

The step 420 may, in some embodiments (e.g., without a WARM(s) 135), be performed entirely by the RAD(s) 165. For example, the RAD(s) 165 may generate an alert message using the alert engine(s) 230 and display the alert on its own user interface (e.g., as shown in FIG. 14). The alert message may, for example, include visual indicators (e.g., highlighting the affected GDZ on the GDZ display 1440), audible alerts (e.g., alarm tones, voice notifications), and/or haptic feedback (e.g., vibration). The alert message may, for example, prompt the user 105 to confirm whether the DIRE is active and/or whether assistance is indicated. The prompt may, for example, include interactive elements such as buttons labeled “I'm OK” and “Send Help.”

In some embodiments, the step 420 may include the RAD(s) 165 automatically initiating additional monitoring. For example, upon detecting a DIRE, the RAD(s) 165 may increase the sampling frequency of physiological sensor input(s) 515 from external wearable sensor(s) 255. The RAD(s) 165 may, for example, activate additional sensors (e.g., GPS for precise location tracking, accelerometer for fall detection). The RAD(s) 165 may, for example, begin recording audio and/or video (if equipped with such capabilities) to provide contextual information to emergency responders.

The decision point 425 may be performed by the RAD(s) 165 monitoring for user input. The RAD(s) 165 may, for example, implement a timeout mechanism. If the user 105 does not respond within a predetermined time period (e.g., 30 seconds, 1 minute, 2 minutes), the RAD(s) 165 may determine that the wearer has not responded and proceed to step 435. The timeout period may, for example, be configurable based on organizational policies and/or the severity of the detected DIRE. For example, a DIRE classified as high severity (e.g., multiple GDZ(s) 115 activated, significant physiological changes) may trigger a shorter timeout period than a DIRE classified as low severity.

In some implementations, the decision point 425 may include monitoring for indirect indicators of user responsiveness. For example, the RAD(s) 165 may monitor physiological sensor input(s) 515 for signs of consciousness (e.g., normal heart rate variability, movement detected by accelerometer). If physiological indicators suggest the user 105 is unconscious or incapacitated, the RAD(s) 165 may immediately proceed to step 435 without waiting for explicit user input. Such embodiments may, for example, advantageously reduce response time in situations where the user 105 is unable to respond.

The decision point 430 may, for example, be performed by the RAD(s) 165 based on user input and/or sensor data. For example, if the user 105 responds by selecting “I'm OK” on the RAD(s) 165 interface, the RAD(s) 165 may prompt the user to confirm that the DIRE was a false alarm. The RAD(s) 165 may, for example, request additional information such as the cause of the false alarm (e.g., “Accidental compression,” “Equipment malfunction,” “Environmental factors”). This information may, for example, be logged and used to increase the DIRE detection engine(s) 225 through machine learning.

In some embodiments, deescalation may require confirmation from sensor data in addition to user input. For example, even if the user 105 indicates that they are OK, the RAD(s) 165 may verify that the affected GDZ(s) 115 has returned to normal pressure readings (if the vacuum seal was not permanently compromised) and/or that physiological parameters have stabilized. If sensor data contradicts the user's self-assessment, the RAD(s) 165 may, for example, generate a warning message and/or proceed with alert escalation despite the user's input. Such embodiments may, for example, advantageously protect users who may underestimate the severity of their condition due to shock, adrenaline, or impaired judgment.

The step 435 may, in some embodiments, be performed by the RAD(s) 165 transmitting alert signals via the communication network(s) 170. The RAD(s) 165 may, for example, generate a DIRE package(s) 530 including comprehensive information about the detected DIRE. The DIRE package(s) 530 may include, by way of example and not limitation, the user's identity (e.g., name, badge number), current GPS coordinates, timestamp of the DIRE, affected GDZ(s) 115 (indicating injury location), physiological data from wearable sensor(s) 255, and/or historical context (e.g., recent activity level, environmental conditions).

In some implementations, the step 435 may include transmitting alerts through multiple channels in parallel, such as to facilitate reliable delivery. For example, the RAD(s) 165 may transmit the DIRE package(s) 530 to the dispatch system(s) 175 via cellular network, to a cloud-based emergency response service (e.g., via RapidSOS API), and/or to nearby RAD(s) 165 worn by other users via peer-to-peer communication. The multi-channel approach may, for example, advantageously provide redundancy in case one communication channel fails or experiences delays.

In some embodiments, the step 435 may include the RAD(s) 165 automatically escalating the alert priority based on the severity of the DIRE and/or lack of user response. For example, if the user 105 fails to respond to multiple alert prompts, the RAD(s) 165 may classify the situation as a high-urgency emergency and transmit high-priority alerts that trigger immediate dispatch of emergency responders. The RAD(s) 165 may, for example, activate additional alert mechanisms such as transmitting distress signals to all nearby RAD(s) 165, activating audible alarms on the RAD(s) 165 itself to attract attention from bystanders, and/or initiating automated voice calls to emergency services.

The decision point 440 may, in some embodiments (e.g., without a WARM(s) 135), be performed by the RAD(s) 165 monitoring for confirmation signals from the dispatch system(s) 175 and/or other recipients of the alert. For example, the dispatch system(s) 175 may transmit an acknowledgment message to the RAD(s) 165 confirming that the alert has been received and that responders have been dispatched. The RAD(s) 165 may, for example, display this confirmation to the user 105 (if conscious) and/or to nearby personnel. The confirmation may include, for example, estimated time of arrival (ETA) for emergency responders and/or instructions for the user 105 or bystanders.

In some implementations, if confirmation is not received within a predetermined time period, the RAD(s) 165 may repeat step 435 with increased urgency. For example, the RAD(s) 165 may attempt to transmit the alert via alternative communication channels (e.g., switching from cellular to satellite communication if cellular coverage is unavailable). The RAD(s) 165 may, for example, increase the frequency of alert transmissions and/or expand the recipient list to include backup dispatch centers, supervisory personnel, and/or emergency contacts designated by the user 105.

In some embodiments, the method 400 may include additional steps (e.g., specific to systems without a WARM(s) 135). For example, the RAD(s) 165 may periodically verify the operational status of each GDZ(s) 115 by querying for status reports. If a GDZ(s) 115 fails to respond to status queries, the RAD(s) 165 may generate a maintenance alert indicating that the GDZ(s) 115 may benefit from inspection, battery replacement, or replacement of the entire unit. Such proactive monitoring may, for example, advantageously ensure system reliability and reduce the risk of undetected failures.

In some implementations, the method 400 may include peer-to-peer alert propagation. For example, if the RAD(s) 165 detects a DIRE and the user 105 fails to respond, the RAD(s) 165 may automatically transmit peer alerts to nearby RAD(s) 165 worn by other users (e.g., fellow officers, team members). The peer alerts may, for example, include the location of the affected user 105 and a request for immediate assistance. Nearby users may, for example, receive visual and/or audible notifications on their own RAD(s) 165 prompting them to provide aid. Such embodiments may, for example, advantageously enable rapid peer response in situations where the affected user is in immediate danger and emergency responders have not yet arrived.

In some embodiments, the method 400 may include automatic activation of emergency features on the RAD(s) 165. For example, upon detecting a DIRE and failing to receive user response, the RAD(s) 165 may automatically activate its camera (if equipped), such as to capture images or video of the scene. The RAD(s) 165 may, for example, activate its microphone, such as to record audio. The captured media may, for example, be automatically transmitted to the dispatch system(s) 175 and/or stored locally for subsequent investigation. Such features may, for example, advantageously provide valuable contextual information to emergency responders and/or investigators.

In some implementations, the method 400 may include coordination with other connected devices. For example, if the user 105 is wearing a body camera, the RAD(s) 165 may communicate with the body camera to trigger recording or to retrieve recent footage. If the user 105 is in a vehicle equipped with telematics, the RAD(s) 165 may communicate with the vehicle system to obtain location data, speed data, and/or crash detection data. Such integration may, for example, advantageously provide a comprehensive picture of the incident to emergency responders.

In some embodiments, the method 400 may include machine learning-based adaptation. For example, the DIRE detection engine(s) 225 may continuously learn from each DIRE event and user response. If a particular type of signal pattern consistently results in false positives that are deescalated by the user, the DIRE detection engine(s) 225 may adjust its detection thresholds to reduce future false positives. Conversely, if certain signal patterns are consistently associated with confirmed DIRE events, the DIRE detection engine(s) 225 may increase its sensitivity to those patterns. Such adaptive learning may, for example, advantageously increase detection accuracy over time and reduce alert fatigue.

In some implementations, the method 400 may include post-incident reporting and analysis. After a DIRE event is resolved (either through deescalation or emergency response), the RAD(s) 165 may generate a comprehensive incident report. The incident report may include, by way of example and not limitation, timeline of events, sensor data (from GDZ(s) 115 and wearable sensor(s) 255), user responses, alerts transmitted, confirmations received, and outcome. The incident report may, for example, be automatically transmitted to a data store(s) 245 for archival and/or analysis. Such reports may, for example, be used for training purposes, system enhancement, and/or compliance with organizational policies and regulations.

FIG. 5 depicts an illustrative block diagram depicting operation and/or training of an example DIRE detection engine. In the depicted example, the DIRE detection engine(s) 225 is configured to receive one or more inputs, and generate a DIRE object 530 in response. As depicted, the inputs include a garment sensor input(s) 510. The garment sensor input(s) may, for example, include inputs from sensor(s) of one or more RAP(s) 110, such as sensor(s) from corresponding GDZ(s) 115. The sensor(s) may, for example, include sensor module(s) 120. A sensor input may, for example, include flex sensor input and/or deformation sensor input. Flex sensor input and/or deformation sensor input may, by way of example and not limitation, advantageously provide data corresponding to flexure of the garment (e.g., GDZ).

A sensor input may, for example, include pressure sensor input. The pressure sensor input may, for example, advantageously provide data corresponding to pressure changes (e.g., pressurization, loss of vacuum) in a garment (e.g., GDZ).

A sensor input may, for example, include motion sensor input. Motion sensor input may, for example, include acceleration data. Motion sensor input may, for example, include speed or velocity data. A motion sensor may, by way of example and not limitation, include an inertial measurement unit (IMU). An IMU may, for example, advantageously provide a compact, single package with multiple motion measurement attributes. A motion sensor may, for example, include an accelerometer. An accelerometer may, for example, advantageously provide acceleration data. A motion sensor may, for example, include a gyrometer. A gyrometer may, for example, advantageously provide rotational motion data. Motion sensor input may, for example, advantageously provide data corresponding to movement of a garment (e.g., GDZ) and/or person.

A sensor input may, for example, include geolocation sensor input. A geolocation sensor may, for example, include a GPS sensor. A GPS sensor may, for example, advantageously provide global navigational coordinate data. A geolocation sensor may, for example, include a triangulation sensor (e.g., including distance measurement sensor(s)). A triangulation sensor may, for example, advantageously provide local navigational data. A geolocation sensor may, for example, include distance measurement sensor(s) (e.g., laser, radar, ultrasonic) and/or proximity sensors. A distance measurement sensor may, for example, advantageously provide environmental location data. A proximity sensor may, for example, advantageously provide immediate (e.g., pre-impact, alignment) environmental positioning data. A distance and/or proximity sensor(s) may, for example, advantageously provide motion sensor input(s) (e.g., multi-function). Geolocation sensor data may, by way of example and not limitation, advantageously provide data corresponding to wearer (e.g., user 105) location and/or movement.

A sensor input may, as shown in the depicted example, include physiological sensor input(s) 515. A physiological sensor may, for example, include temperature sensor input(s). Temperature sensor input(s) may, for example, advantageously provide data corresponding to body temperature. Temperature sensor input(s) may, for example, advantageously provide data corresponding to skin temperature. Temperature sensor input(s) may, for example, advantageously provide data corresponding to environmental temperature. Temperature sensor(s) may, for example, be contact based. Contact-based sensor(s) may, for example, provide high-resolution and/or low power. Temperature sensor(s) may, for example, be contactless. Contactless temperature sensor(s) may, for example, advantageously monitor temperature of regions inconvenient to remain in contact.

A physiological sensor input may, for example, include blood pressure sensor input(s). Blood pressure sensor input(s) may, for example, advantageously provide data corresponding to blood pressure of a wearer. Blood pressure may, for example, advantageously provide insight into current physical, mental, and/or emotional events of the wearer.

A physiological sensor input may, for example, include electrical field sensor input(s). Electroencephalogram (EEG) sensor input(s) may, for example, advantageously provide data corresponding to brain activity of a wearer. Electrocardiogram (ECG) sensor input(s) may, for example, advantageously provide data corresponding to heart activity of a wearer. Electromyogram (EMG) sensor input(s) may, for example, advantageously provide data corresponding to muscle activity (e.g., muscle tone, muscle actuation). Electrooculograph (EOG) sensor input(s) may, for example, advantageously provide data corresponding to eye activity. Electrodermograph (EDG) sensor input(s) may, for example, advantageously provide data corresponding to skin changes.

A physiological sensor input may, for example, include microelectromechanical (MEMS) sensors. MEMS sensors may, for example, advantageously provide data corresponding to physical quantities like pressure, acceleration, temperature, and/or magnetic fields.

A physiological sensor input may, for example, include biological and/or chemical sensor(s). For example, a biological and/or chemical sensor input(s) may include blood glucose data. Blood glucose data may, for example, advantageously provide insight into a wearer's disorder status (e.g., diabetes) and/or energy level (e.g., hypoglycemia). Sensor input(s) may, for example, include analyte levels, such as sodium. A sodium sensor may, for example, advantageously provide data corresponding to hydration level. In some implementations, by way of example and not limitation, a sensor input(s) may include hormone and/or other biomarker data. Hormone excretion data may, for example, advantageously provide insight into stress level and/or other situational and/or physiological data.

In the depicted embodiment, inputs include, by way of example and not limitation, historical input(s) 520. Historical input(s) may include, as depicted, historical DIRE objects. Historical input(s) may, as depicted, include historical input(s) associated with corresponding historical DIRE objects. Associated inputs may, for example, include weather data. Associated inputs may, for example, include physiological data. Associated inputs may, for example, include geolocation data. Associated inputs may, for example, include situational data. Associated inputs may, for example, include generated outputs and/or responses to and/or corresponding to the DIRE. Associated inputs may, for example, include one or more outcome(s) corresponding to the historical DIRE(s). For example, historical data associated with corresponding DIRE(s) may advantageously enable training of the DIRE detection engine(s) 225.

In the depicted example, inputs include, by way of example and not limitation, historical RAP input(s) 525. Historical RAP input(s) may, for example, include GDZ object(s). Historical RAP input(s) 525 may, for example, include historical garment sensor input(s), as shown in the depicted example. The historical garment sensor input(s) (e.g., garment sensor input(s) 510). The historical RAP input(s) 525 may, for example, include historical physiological sensor input(s). The historical sensor input(s) may, for example, be selected based on association with the historical activated GDZ object(s). The historical RAP input(s) may, for example, include associations with historical input(s) 520. For example, the historical RAP input(s) 525 may be selected based on association with historical input(s) 520. The historical input(s) 520 may, for example, be selected based on association with the historical RAP input(s) 525. The historical RAP input(s) 525 may, for example, advantageously enable training of the DIRE detection engine(s) 225. For example, the historical RAP input(s) 525 and/or historical input(s) 520 may advantageously enable training of the DIRE detection engine(s) 225 to identify potential outcomes based on fusion of garment sensor input(s) 510 (e.g., GDZ data, other data discussed above) and/or physiological sensor input(s) 515 to more accurately identify, classify, and/or assess potential DIRE(s).

The DIRE object(s) 530 may, for example, be generated based on the garment sensor input(s) 510 and/or physiological sensor input(s) 515. In some implementations, the DIRE object(s) 530 may be generated based on the historical input(s) 520 and/or historical RAP input(s) 525. In some examples, the DIRE detection engine(s) 225 may be trained using the historical input(s) 520 and/or historical RAP input(s) 525 in an iterative process such as, by way of example and not limitation, disclosed at least with reference to FIG. 3.

In some implementations, one or more sensors may, for example, be self-powered. In some embodiments, sensor(s) and/or other components may, by way of example and not limitation, be powered (e.g., partially, completely) by motion and/or by the wearer. In some examples, one or more components may be at least partially powered by a connected WARM(s) 135.

FIG. 6 depicts an example garment detection zone (GDZ) of a RAP in an embodiment including an internal pressure detection sensor. In the depicted example, the GDZ(s) 115 includes boundary wall(s) 150. The boundary wall(s) 150 enclose an interior region(s) 155. In this embodiment, a sensor module 605 (e.g., sensor module(s) 120) is disposed within the interior region(s) 155. For example, the sensor module(s) 605 may be enclosed within the interior region(s) 155. The sensor module(s) 605 may, for example, include a pressure sensor. The sensor module(s) 605 may, for example, detect when the pressure of the interior region(s) 155 changes. As shown, the sensor module(s) 605 is communicably coupled (e.g., electrically coupled, optically coupled) to a communication channel(s) 125.

By way of example and not limitation, the boundary wall(s) 150 may be configured as a vacuum sealed bladder containing an internal miniature air pressure sensor (e.g., sensor module(s) 605) configured to measure pressure (e.g., absolute pressure). A bladder may, for example, be sized according to a desired detection zone (e.g., GDZ). For example, bladders for different body regions may be configured with different sizes (e.g., according to target spatial resolution of detection). In some implementations, bladders may be sized uniformly for a given RAP. Bladders may, for example, be inserted into pockets and/or other cavities within a RAP. In some implementations, the communication channel(s) 125 may be integrated (e.g., woven, bonded, adhered) to the RAP. The sensor module(s) 605 may, for example, make electrical contact with the communication channel(s) 125 when inserted into the cavity. The RAP may, for example, be configured as a base layer (e.g., undergarment).

When sealed and vacuumed, the sensor module(s) 605 may, by way of example and not limitation, read between 0-3 kPa. Upon puncture of the boundary wall(s) 150, the inrush of air may, for example, raise the pressure readings, eventually settling close to atmospheric pressure. The change in pressure may, for example, result in the WARM(s) 135 and/or RAD(s) 165 detecting a DIRE.

FIG. 7 depicts an example GDZ of a RAP in an embodiment including an expanding media and an electric attribute detection module. In the depicted example, the GDZ(s) 115 includes boundary wall(s) 150. The boundary wall(s) 150 enclose an interior region(s) 155. In this embodiment, expandable media 705 is disposed within the interior region(s) 155. For example, the expandable media 705 may be operated into a compressed state having a first volume. In the presence of higher pressure (e.g., elevation from sub-atmospheric to atmospheric pressure, such as if the interior region(s) 155 is under vacuum and the boundary wall(s) 150 is then penetrated), the expandable media 705 may self-expand. The expansion may, for example, change one or more attributes measured by a sensor module (e.g., sensor module(s) 120). The sensor module may, as shown in the depicted example, be configured to measure an electric attribute(s). For example, the sensor module may include, as depicted, one or more cathode(s) 710 and one or more anode(s) 715. The expansion of the expandable media 705 may, for example, result in a change in current between the cathode(s) 710 and the anode(s) 715. The change in current may, for example, be detected (e.g., by the WARM(s) 135 and/or RAD(s) 165) as a GDZ penetration.

As an illustrative example, the expandable media 705 may be configured as a thin layer of compressible poly(vinyl alcohol)-glycerol (PVA-glycerol) hydrogel within a vacuum seal bag (e.g., boundary wall(s) 150). Pressure applied to the gel may, for example, produce a current across a cathode (e.g., cathode(s) 710) and anode (e.g., anode(s) 715). The cathode may, by way of example and not limitation, be at least partially constructed from MXene. The anode may, by way of example and not limitation, be at least partially constructed of aluminum. The current may, for example, scale linearly with pressure changes. Upon puncture, the gel may, for example, expand due to atmospheric pressure, changing the observed current and triggering an alarm (e.g., as disclosed at least with reference to FIG. 4).

FIG. 8 depicts an example GDZ of a RAP in an embodiment including a piezoelectric film. As depicted, the boundary wall(s) 150 encloses an interior region(s) 155. A sensor module 805 is disposed along at least surface of the boundary wall(s) 150. In the depicted example, the sensor module(s) 805 is disposed within the interior region(s) 155 on an interior surface of the boundary wall(s) 150. The sensor module(s) 805 is communicably coupled to a communication channel(s) 125. The sensor module(s) 805 may, for example, be configured as a film. The film may, by way of example and not limitation, include a piezoelectric film. The sensor module(s) 805 may, for example, be bonded (e.g., adhesively, by fasteners, welded) to the boundary wall(s) 150.

As an illustrative example, the sensor module(s) 805 may be a piezoelectric film. The piezoelectric film may, for example, be configured to generate signals corresponding to pressure changes within a vacuum seal bag (e.g., the GDZ(s) 115). For example, an impedance of the sensor module(s) 805 may change in response to a change in pressure. A reference impedance may, for example, be obtained under vacuum (e.g., in a configuration method such as disclosed at least with reference to FIG. 3). Upon puncture and exposure to atmospheric pressure, the change in impedance may, for example, indicate penetration of the GDZ(s) 115.

Although FIGS. 6-8 depict physical communication channels, such embodiments may be configured with wireless communication channels. For example, The GDZ(s) 115 may include a communication module(s). The communication module(s) may establish a wireless communication channel(s) 125, such as, by way of example and not limitation, with a WARM(s), RAD(s), and/or other GDZ(s).

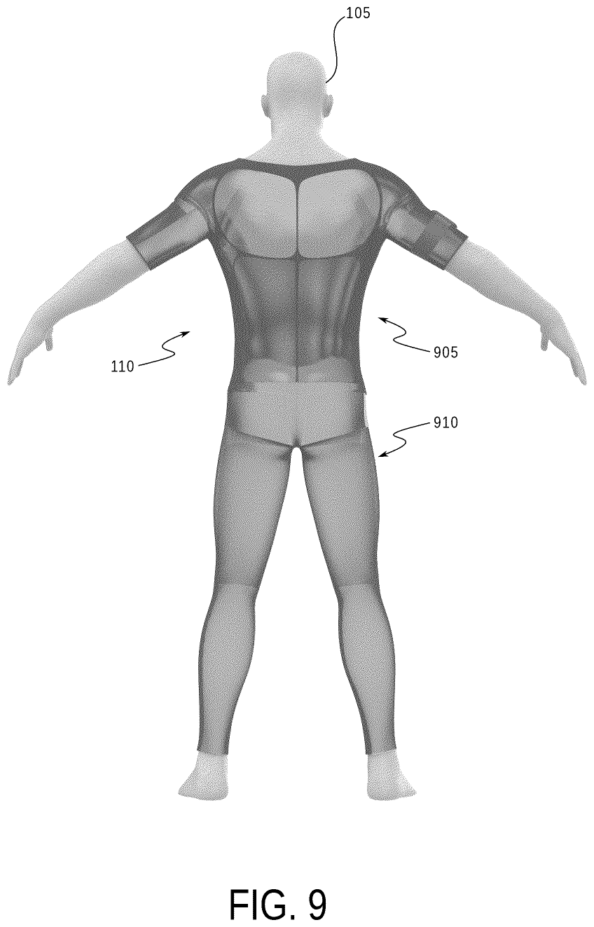

FIG. 9 depicts an illustrative embodiment including an upper RAP 905 and a lower RAP 910. Some embodiments, such as depicted, may include multiple RAPs. The RAPs may, for example, be functionally independent. For example, each RAP may couple to one or more (e.g., own) WARM(s) 135. Such embodiments may, for example, advantageously provide rapid interchangeability and/or increased redundancy.

Some embodiments may, for example, interconnect. For example, the upper RAP(s) 905 may communicably couple to the lower RAP(s) 910. Each RAP may, for example, include a communication interface. The communication harness may, for example, include a wire harness. The user 105 may, for example, couple the wire harness (e.g., after donning the upper RAP(s) 905 and lower RAP(s) 910). In some implementations, the upper RAP(s) 905 and lower RAP(s) 910 may, for example, be self-connecting. For example, the upper RAP(s) 905 and lower RAP(s) 910 may include wireless communication modules. The wireless communication modules may be self-pairing (e.g., by BLUETOOTH low energy). The wireless communication modules may, for example, be paired manually. The wireless communication modules may, for example, enable freedom of movement of the user 105 without disrupting communication between the connected RAPs. Paired RAPs may, for example, advantageously reduce the number of WARM(s) 135.

In some implementations, the upper RAP(s) 905 and lower RAP(s) 910 may include self-connecting contacts. For example, the upper RAP(s) 905 and lower RAP(s) 910 may fasten together (e.g., buttons, hook-and-loop, snaps, magnets). The communication interface(s) may, for example, be brought into alignment and communicably coupled when the fasteners are coupled together. Accordingly, the various RAP(s) may, for example, advantageously be communicably coupled when mechanically coupled together.

FIG. 10 and FIG. 11 depict an example releasably coupled WARM. In the depicted example, the WARM(s) 135 includes a user interface 1005. The user interface(s) 1005 may, for example, include one or more button and/or other touch input(s). The user interface(s) 1005 may, for example, be configured to operate the WARM(s) 135 into a mode (e.g., power-on, active, power-off, disabled), such as by a user 105. In the event of an alert message (e.g., in response to a DIRE, such as disclosed at least with reference to steps 420-430 of FIG. 4), the user interface(s) 1005 may enable the user 105 to deescalate a DIRE. For example, the user interface(s) 1005 may advantageously enable the WARM(s) 135 to operate as a RAD.

In some embodiments, the user interface(s) 1005 may include a graphical display. The graphical display may, for example, display a status of the user interface(s) 1005 and/or information related to a warning (e.g., DIRE).

In some implementations, the WARM(s) 135 may include, for example, multiple user interface(s) 1005. For example, the WARM(s) 135 may include multiple (e.g., distinct) buttons and/or other touch inputs (e.g., capacitive touch point, resistive touch point). The WARM(s) 135 may include, for example, touch input and one or more visual indicator(s) (e.g., graphical user display).

In this example, the WARM(s) 135 includes a communication port 1105. The communication port 1105 may include, for example, provide a connection port to a communication module(s) 240. The communication port 1105 may, for example, be configured as USB-C connector. The communication port 1105 may, for example, transmit power and/or data between (e.g., from, to) the WARM(s) 135 and external device(s).

The WARM(s) 135 includes, in the depicted example, a sensor 1110 (e.g., physiological sensor). The sensor(s) 1110 may, for example, include a photoplethysmogram (PPG) sensor. The PPG sensor may, for example, be configured to detect blood volume changes in the microvascular bed of tissue. For example, the PPG may be configured as pulse oximeter illuminating the skin and measuring changes in light absorption. The PPG may, for example, advantageously enable monitoring of the perfusion of blood to the dermis and subcutaneous tissue of the skin. The sensor(s) 1110 may, for example, generate physiological sensor input(s) 515. In some embodiments, data from the PPG may be used to generate blood oxygen (e.g., SpO2) measurement(s).

In this example, the WARM(s) 135 includes a temperature sensor 1115. The temperature sensor(s) 115 may, for example generate physiological sensor input(s) 515. The temperature sensor(s) 115 may, for example, advantageously monitor temperature of the user 105.

in the depicted example, the WARM(s) 135 includes contacts 1120. The contact(s) 1120 may, for example, be spring-loaded contact pins. The contact(s) 1120 may, for example transmit data and/or power to and/or from the WARM(s) 135. For example, the contact(s) 1120 may be configured as a secondary charging connectors (e.g., to charge the power storage module(s) 250). The contact(s) 1120 may, for example, be configured to communicably couple to the coupling module(s) 130.

The WARM(s) 135 includes, in this example, coupling modules 1125. For example, as depicted, the coupling modules 1125 may be configured as protrusions configured to engage mating cavities and/or protrusions. Engagement of the coupling modules 1125 may, for example, releasably couple the WARM(s) 135 to a device (e.g., the coupling module(s) 130).

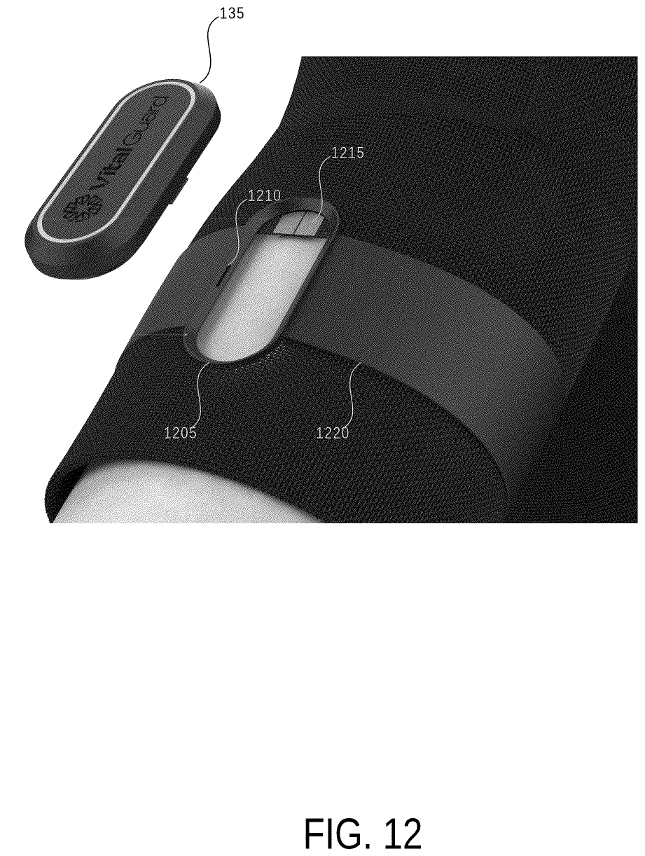

FIG. 12 depicts an example WARM, such as depicted in FIGS. 10-11, and a corresponding coupling module in an example garment (e.g., RAP). The RAP(s) 110 includes a mechanical interface 1205 configured to align with the WARM(s) 135. In the depicted example, the mechanical interface(s) 1205 includes an aperture with a contoured (e.g., tapered) rim configured to align with the perimeter of the WARM(s) 135.

The mechanical interface(s) 1205 includes a coupling module 1210 (e.g., on opposing sides of the mechanical interface(s) 1205). The coupling module(s) 1210 may, for example, be configured to align and matingly couple with the coupling modules 1125 of the WARM(s) 135. For example, the coupling module(s) 1210 and coupling modules 1125 may cooperate to releasably couple the WARM(s) 135 to the mechanical interface(s) 1205.

The RAP(s) 110 includes a communication interface 1215. The communication interface(s) 1215 may, for example, be positioned and configured to automatically communicably couple to the WARM(s) 135, such as via the contact(s) 1120. For example, the communication interface(s) 1215 may be configured as at least a portion of the coupling module(s) 130. The contact(s) 1120 may be communicably coupled to one or more GDZs (e.g., GDZ(s) 115) via one or more corresponding communication channel(s) 125.

In this example, the mechanical interface(s) 1205 is coupled to the user 105 and/or the RAP(s) 110 via a coupling module 1220. As depicted, the coupling module(s) 1220 is configured as an armband, for example. The coupling module(s) 1220 may, for example, advantageously prevent accidental dislodgement of the mechanical interface(s) 1205 from the RAP(s) 110.

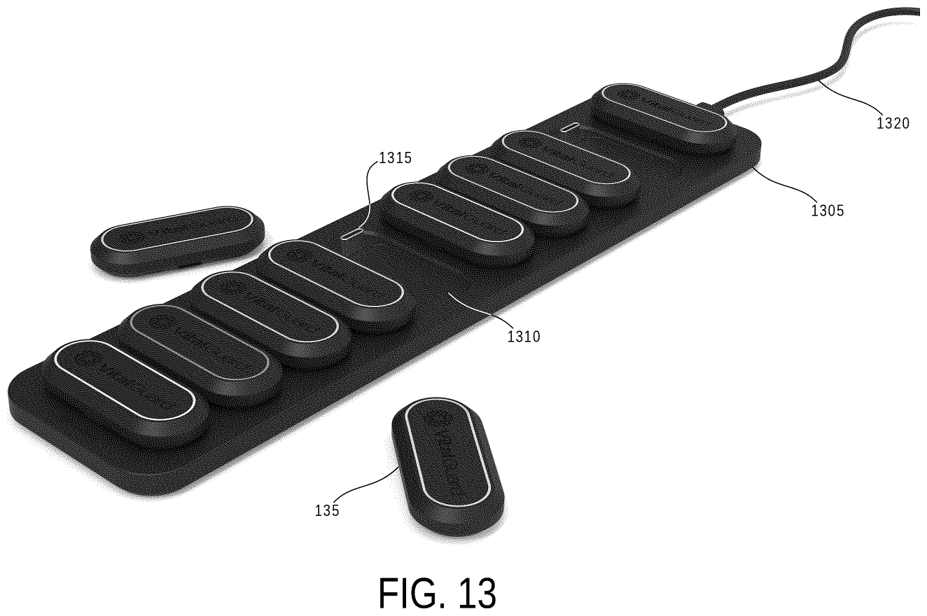

FIG. 13 depicts an example multi-WARM docking station. A docking station 1305 may, for example, be provided with multiple ports 1310. Each port(s) 1310 may, for example, be configured to receive a corresponding WARM(s) 135. In the depicted example, each port(s) 1310 is provided with a I/O port 1315. For example, the I/O port(s) 1315 may include a USB-C port, as depicted. The I/O port(s) 1315 may, for example, be configured to align with and communicably couple to the WARM(s) 135 via the communication port 1105. The docking station 1305, in this example, includes a communication and/or power link 1320 (e.g., a cable, as shown). The power link(s) 1320 may, for example, pluggably couple the docking station 1305 to a device, network, and/or power source.
in some implementations, the docking station 1305 may be configured as a charging station. The docking station 1305 may, for example, be configured as a data transfer (e.g., syncing) station. As an illustrative example, a user 105 may, at the end of use of a RAP(s) 110 (e.g., at the end of a shift), remove the WARM(s) 135(s) from the RAP(s) 110 and couple it to the docking station 1305. In preparation for using a RAP(s) 110 (e.g., at the beginning of a shift, upon or before donning the RAP(s) 110), a user 105 may decouple a WARM(s) 135 from the docking station 1305 and couple it to the RAP(s) 110. In some implementations, a WARM(s) 135 may, for example, be assigned to a specific user and/or RAP. Such embodiments may, for example, advantageously reduce power and/or time in configuration for a specific RAP and/or user. In some implementations, WARM(s) 135 may be interchangeable. Such embodiments may advantageously allow a user to grab any available charged WARM(s) 135. For example, the WARM(s) 135 may automatically configure itself upon use (e.g., as disclosed at least with reference to FIG. 3).

FIG. 14 depicts an illustrative user interface (UI) of an example RAD. In the depicted example, the UI includes a physiological display 1405. As depicted, the weapon alert message(s) 1450 includes a heart rate display 1420 (e.g., from an EEG sensor). The weapon alert message(s) 1450 includes, in this example, a body temperature display 1425 (e.g., from the temperature sensor(s) 115). The weapon alert message(s) 1450 includes, in the depicted example, a blood oxygenation display 1430 (e.g., from the sensor(s) 1110). The weapon alert message(s) 1450 includes, in the depicted example, a calories burned display 1435. The calories burned display(s) 1435 may, for example, be calculated based on data from a motion sensor(s) (e.g., embedded, from a connected wearable device such as a smartwatch, from the RAD(s) 165 itself).

The UI includes, in this example, a DIRE location display 1410. The DIRE location display(s) 1410 includes a GDZ display 1440. The GDZ display(s) 1440 may, for example, depict the GDZs of the RAP(s) coupled to the currently connected WARM(s) 135. The GDZ display(s) 1440 may be configured, for example, to display GDZ(s) detected as involved in a DIRE. For example, as depicted, the GDZ display(s) 1440 is displaying active GDZs 1445. A DIRE messaging display 1415 displays further information about the DIRE corresponding to the active GDZ(s) 1445. The DIRE messaging display(s) 1415 includes, in the depicted example, an alert message weapon 1450, and a DIRE status weapon 1455. Accordingly, the RAD(s) 165 may advantageously communicate to a user (e.g., user 105, dispatcher, manager, fellow responder) the physiological and/or DIRE status of a current RAP(s).

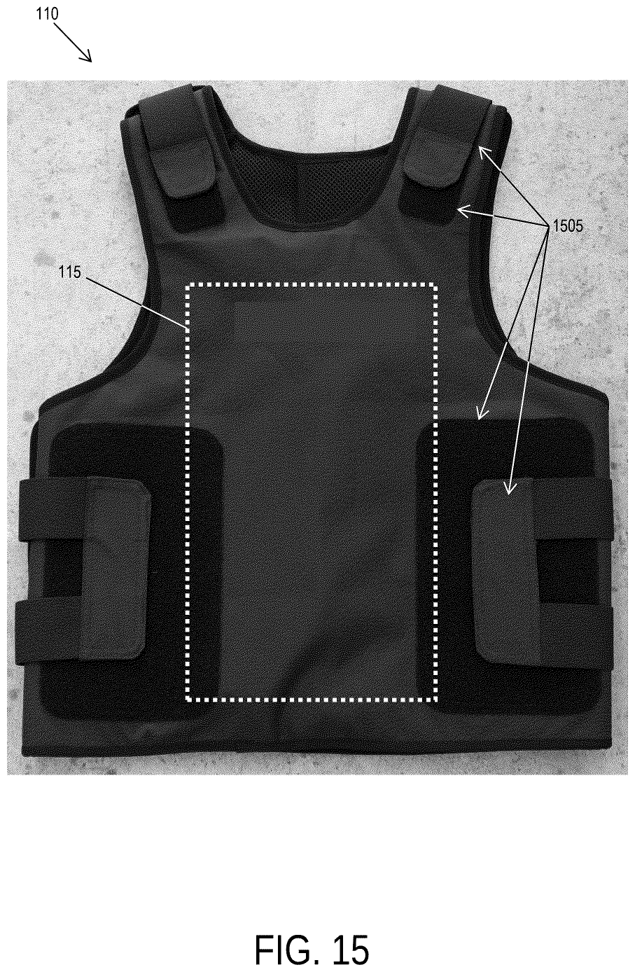

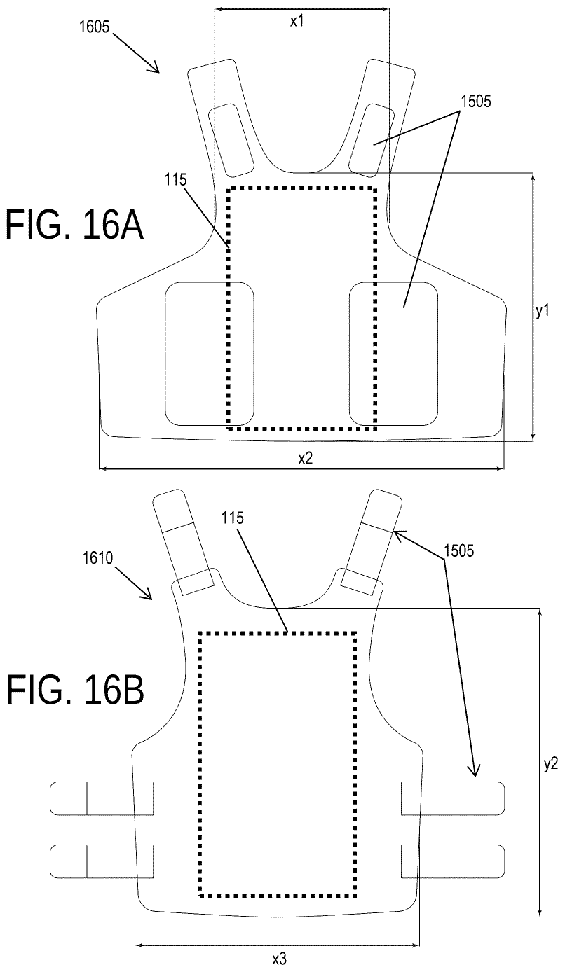

FIG. 15, FIG. 16A, and FIG. 16B depict an example embodiment(s) of an example responsive apportioned garment (RAP 110) configured as a vest, including, by way of example and not limitation, wirelessly communicable garment detection zones (GDZs 115). In the depicted example, the RAP 110 is configured as a 2-piece vest. A first portion 1605 may, for example, be configured as a front half (e.g., over a user's torso front). A second portion 1610 may, for example, be configured as a rear half (e.g., over a user's back).

In this example, portions (e.g., first portion 1605 and second portion 1610) are coupled together by coupling modules 1505. As shown, the coupling modules are embodied as complementary hook-and-loop fabric patches. Some embodiments may, for example, be configured with various releasable coupling mechanisms. For example, some embodiments may include rigid hooks interdigitating with loops. Some embodiments may, for example, include snaps and/or buttons. Some embodiments may, for example, include ties. Some embodiments may, for example, include latches.

In the depicted example, the RAP 110 includes a single GDZ 115 in the first portion 1605. The depicted RAP 110 includes a single GDZ 115 in the rear portion 1605. A reduced number of GDZs may, for example, advantageously reduce cost and/or complexity. The reduced number may, for example, advantageously increase reliability (e.g., by reducing complexity). Some embodiments may, for example, include a single GDZ (e.g., wrapped around front and back). Some embodiments may, as disclosed with respect to other embodiments, include multiple GDZs.

In some embodiments, the dimensions of the first portion 1605 and second portion 1610 may be selected to provide proper fitment across various body sizes and/or armor carrier configurations. As depicted in FIG. 16A, the first portion 1605 may be characterized by a width dimension x1 and a height dimension y1. The width dimension x1 may, for example, extend laterally across the front torso region. The height dimension y1 may, for example, extend vertically from an upper chest region to a lower abdomen region. In some implementations, the width dimension x1 may range from approximately 10 inches to approximately 18 inches. The height dimension y1 may, for example, range from approximately 12 inches to approximately 20 inches. Such dimensions may, for example, advantageously provide coverage of vital organs including the heart, lungs, and/or major blood vessels in the torso.

As depicted in FIG. 16B, the second portion 1610 may be characterized by a width dimension x2 and a height dimension y2. The width dimension x2 may, for example, extend laterally across the rear torso region. The height dimension y2 may, for example, extend vertically from an upper back region to a lower back region. In some implementations, the width dimension x2 may be substantially similar to the width dimension x1 (e.g., within 10%, within 5%, substantially equal). For example, the width dimension x2 may range from approximately 10 inches to approximately 18 inches. The height dimension y2 may, for example, be substantially similar to the height dimension y1. In some embodiments, the height dimension y2 may differ from the height dimension y 1 to accommodate anatomical differences between the front and rear torso. For example, the height dimension y2 may be slightly greater than the height dimension y1 to provide extended coverage of the spine and/or kidney regions.

The coupling modules 1505 may be positioned to span a lateral distance x3, as depicted. The lateral distance x3 may, for example, represent the separation between the coupling modules 1505 on opposing sides of the RAP 110. In some implementations, the lateral distance x3 may be selected to accommodate the circumference of the user's torso. For example, the lateral distance x3 may range from approximately 12 inches to approximately 24 inches when the first portion 1605 and second portion 1610 are coupled together. The adjustability provided by the coupling modules 1505 (e.g., hook-and-loop fasteners) may, for example, enable the lateral distance x3 to be varied to achieve proper fit. Such adjustability may, for example, advantageously enable a single size RAP 110 to accommodate a range of torso circumferences (e.g., 32 inches to 48 inches).

In some embodiments, the dimensions x1, x2, y1, and y2 may be scaled proportionally to create multiple size variants. For example, a small size RAP may have dimensions x1 and x2 of approximately 10-12 inches and dimensions y 1 and y2 of approximately 12-14 inches. A medium size RAP may, for example, have dimensions x1 and x2 of approximately 13-15 inches and dimensions y1 and y2 of approximately 15-17 inches. A large size RAP may, for example, have dimensions x1 and x2 of approximately 16-18 inches and dimensions y1 and y2 of approximately 18-20 inches. Such standardized sizing may, for example, advantageously simplify inventory management and/or procurement decisions while providing adequate fit across a user population.

The dimensions may, for example, be selected based on anthropometric data. Anthropometric data may include, by way of example and not limitation, torso width measurements, torso height measurements, and/or chest circumference measurements from target user populations (e.g., law enforcement officers, military personnel, first responders). In some implementations, the dimensions may be selected to provide coverage of a predetermined percentage of the target population (e.g., 90%, 95%, 99%). For example, the dimensions may be selected such that the GDZ(s) 115 cover vital anatomical regions for users ranging from the 5th percentile female to the 95th percentile male in the target population.

In some embodiments, the GDZ(s) 115 within the first portion 1605 and second portion 1610 may have dimensions slightly smaller than the overall dimensions x1, y1, x2, and y2 to provide clearance for seams, fasteners, and/or edge finishing. For example, the GDZ(s) 115 in the first portion 1605 may have a width of (x1-1 inch) and a height of (y1-1 inch). Such clearance may, for example, advantageously prevent damage to the GDZ(s) 115 during manufacturing, donning, and/or use. The clearance may, for example, provide space for the boundary wall(s) 150 to be securely attached to the surrounding garment material without compromising the vacuum seal integrity.

The aspect ratio of the GDZ(s) 115 (e.g., the ratio of width to height) may, for example, be selected to achieve a target detection coverage and/or manufacturing efficiency. In some implementations, the aspect ratio may range from approximately 0.5 to approximately 1.5. An aspect ratio closer to 1.0 (e.g., approximately square) may, for example, advantageously provide uniform coverage in both lateral and vertical directions. An aspect ratio greater than 1.0 (e.g., wider than tall) may, for example, advantageously provide extended lateral coverage, such as for protection against side impacts. An aspect ratio less than 1.0 (e.g., taller than wide) may, for example, advantageously provide extended vertical coverage, such as for protection of the full torso length.

In some embodiments, the dimensions may be selected to enable compatibility with standard ballistic plate carriers and/or body armor systems. For example, the dimensions x1, y1, x2, and y2 may correspond to standard plate sizes (e.g., 10″×12″, 11″×14″) used in law enforcement and/or military applications. Such compatibility may, for example, advantageously enable the RAP 110 to be worn in conjunction with existing armor systems without mandating modification of the armor carrier. The RAP 110 may, for example, be configured to fit within the same pockets or cavities that normally receive ballistic plates, thereby providing impact detection capability without altering the user's existing equipment configuration.

Some embodiments of RAPs may, by way of example and not limitation, include overlapping GDZs. Overlapping RAPs may, for example, increase reliability of detection (e.g., by providing redundancy, such as in vital areas).

Some embodiments may include GDZs with wireless modules. The wireless modules may, for example, include printed circuit boards (PCBs). For example, the communication channel(s) may be wireless. The wireless design may, for example, facilitate maintaining vacuum seal integrity. For example, wireless GDZs (e.g., individually wirelessly coupled to a wearable automatic response module (WARM) and/or remote alert device (RAD)) may advantageously address challenges associated with traditional impact detection systems. For example, traditional systems may be difficult to maintain to function with vacuum-sealed GDZs due to wired connections compromising the vacuum seal.

In some examples, the GDZs may, for example, include pressure sensors. The pressure sensors may, for example, be positioned at the front and back. For example, a single PCB may include the communication module(s) (e.g., wireless module(s)) and sensor(s) for a given GDZ. These pressure sensors may, for example, monitor the vacuum environment (e.g., within the GDZ). The pressure sensors may, for example, be configured such that a controller (e.g., on the PCB) communicably coupled to the sensor(s) (e.g., the PCB for the GDZ) may perform substantially continuous pressure monitoring (e.g., polled at<1 min frequency, sampled at 1 Hz frequency, sampled greater than 1 Hz). Continuous pressure monitoring may, for example, detect sudden changes.

A housing (e.g., plastic such as 3D printed, molded, formed) may, for example, protect the electronics. This housing may, for example, offer flexibility in design and material selection.

In some embodiments, the wireless module(s) may include a Bluetooth communication system. The Bluetooth communication system may, for example, facilitate wireless data transmission. In some examples, Bluetooth communication may be coded. Coded Bluetooth signals may, for example, achieve secure communication.

A main communication module may, for example, include satellite, cellular, and/or GPS capabilities. For example, the main communication module may be configured to communicate with a RAD(s). In some embodiments, the individual GDZs may communicate with a RAD (e.g., directly, via a WARM).

Some embodiments may, for example, include a battery-powered system. The battery-powered system may, for example, have a long battery life (e.g., 6 months or more). In some examples, circular batteries may power the PCBs. A rechargeable battery may, for example, power a main module.

In some examples, minor pressure variations may be ignored. In some examples, sudden pressure changes may be detected. PCBs may, for example, be positioned 1-2 feet from the communication module. In some examples, the system may identify the location of a breach. The breach may, for example, be at the front and/or back. These features may, for example, provide an immediate alert system for a DIRE (e.g., puncture detection).

In some examples, coverage may be achieved with just two PCB units. In some embodiments, different types of Bluetooth modules may be used. In some examples, various 3D printing materials may be employed for housing. Some embodiments may include various battery configurations. These features may offer flexibility in design and material selection.

Some embodiments may include a RAP with a wireless communication system. The wireless communication system may, for example, utilize NFC technology. NFC technology may, for example, facilitate data transmission. In some examples, PCBs within a vacuum detection bag, such as a GDZ, may send data to a communication module via Bluetooth. Wireless configurations may, for example, reduce physical connections. Reducing physical connections may, for example, enhance the product's flexibility. In some examples, this configuration may facilitate ease of use. The configuration may, for example, increase the longevity of a vacuum seal. In some examples, the configuration may enhance the reliability of a vacuum seal. Manufacturing costs of a vacuum-sealed GDZ may, for example, be reduced.

In some embodiments, each impact section may function as a standalone unit. These standalone units may, for example, operate independently. In some examples, the units may not rely on wires to connect them. This configuration may, for example, allow for modularity. Modularity may, for example, facilitate ease of maintenance. In some examples, each unit can be individually serviced or replaced.

In some embodiments, the product may be designed in a vest format. This vest format may, for example, resemble body armor. Such a form factor change may, for example, provide a more ergonomic fit. An ergonomic fit may, for example, increase comfort for the user. Increased comfort may be particularly advantageous in applications relying on mobility.

In some embodiments, the product may be configured for both armored and unarmored applications. This versatile design may, for example, allow the product to be used in a variety of scenarios. Using the product in various scenarios may, for example, provide flexibility to the user. Flexibility may, for example, depend on the level of protection targeted.

In some embodiments, the impact detection technology may be adaptable to various forms. This adaptability may, for example, allow the technology to be integrated into different types of protective gear or equipment. Integrating the technology into different types of gear may, for example, enhance the product's applicability. Applicability may, for example, extend across different industries or use cases.

In some implementations, garments may, for example, be retrofitted into a RAP(s). For example, GDZs (e.g., GDZ(s) 115) may be coupled (e.g., fastened, adhered, releasably coupled) to existing garments (e.g., vests, headgear, outer garments, under garments, armor, accessories such as backpacks). For example, the GDZs may, for example, be coupled to a coupling module(s) 130 (e.g., via one or more communication channel(s) 125).

For example, some embodiments may be implemented in a military and/or war fighter environment. A RAP(s) may, for example, be configured as ballistic armor and/or other protective gear.

In some examples, a RAP(s) may be configured for an industrial environment. For example, a RAP may be configured as personal protective equipment (PPE) for industrial workers. A miner may, for example, be provided with a RAP(s). A machine operator may, for example, be provided with a RAP(s). The RAP(s) system(s) (e.g., RAP(s) 110, WARM(s) 135, RAD(s) 165) may, for example, be configured with specific sensor(s) and/or logic for the specific industrial environment. For example, DIRE detection logic and/or threshold(s) may be adjusted based on an expected environment and associated threat profile(s).

In some implementations, a RAP(s) 110 may be configured for a healthcare environment. For example, a RAP(s) may be configured to monitor a recovering patient and/or person at heightened risk (e.g., of fall). GDZ(s) of the RAP may, for example, be configured to break when subjected to a specific pressure and/or environment (e.g., excessive moisture, such as by a timed dissolving of at least a portion of a GDZ wall). Such embodiments may, for example, advantageously enable patient monitoring, such as in a hospital, clinic, and/or remote healthcare setting.

In some embodiments, the RAD(s) 165, WARM(s) 135, and/or GDZ(s) 115 may be configured to integrate with third-party emergency response application programming interfaces (APIs). For example, the system may be configured to communicate with emergency routing services such as RAPIDSOS. The APIs may, for example, enable direct communication with emergency dispatch centers (e.g., 911 Public Safety Answering Points). Such integration may, for example, advantageously provide enhanced location accuracy and/or supplemental data transmission to emergency responders. The API integration may, for example, transmit DIRE package(s) including location data (e.g., GPS coordinates), physiological data, and/or injury localization information directly to dispatch systems. In some implementations, the API may enable bidirectional communication, allowing dispatch personnel to send acknowledgment signals and/or request additional information from the RAD(s) 165.

Although various embodiments are disclosed with respect to connection of a GDZ(s) through a WARM and/or RAD, other embodiments are contemplated. For example, in some embodiments, each GDZ(s) 115 may be configured to operate as a standalone unit. A standalone GDZ(s) 115 may, for example, include sufficient components for independent operation. Such components may, for example, include sensor(s) 255, communication module(s) 240, power storage module(s) 250, and processing capability (e.g., a microcontroller, processor). The standalone GDZ(s) 115 may, by way of example and not limitation, be configured to detect a DIRE independently, such as without communication with other GDZ(s) 115 and/or intermediate devices (e.g., WARM(s) 135). Such embodiments may, for example, advantageously provide increased reliability through redundancy, as failure of one GDZ(s) 115 may not compromise the functionality of other GDZ(s) 115.

In some embodiments, standalone GDZ(s) 115 may, for example, be configured to communicate directly with a RAD(s) 165 and/or dispatch system(s) 175. For example, each standalone GDZ(s) 115 may establish an independent wireless connection (e.g., Bluetooth, Wi-Fi, cellular) to the RAD(s) 165. The RAD(s) 165 may, for example, be configured to receive signals from multiple standalone GDZ(s) 115 in parallel and aggregate the data for DIRE detection and/or injury localization. In some implementations, the standalone GDZ(s) 115 may include onboard DIRE detection capability (e.g., a DIRE detection engine(s) 225 implemented in firmware or software on the GDZ's processor). Such embodiments may, for example, advantageously enable the GDZ(s) 115 to generate DIRE package(s) 530 and/or alert package(s) independently, reducing latency and/or computational load on the RAD(s) 165.

Standalone operation may, for example, advantageously simplify system architecture, such as by reducing dependencies on centralized communication hubs and/or intermediate processing modules. The modular nature of standalone GDZ(s) 115 may, for example, facilitate rapid deployment, replacement, and/or maintenance. For example, a damaged or depleted GDZ(s) 115 may be removed and replaced without affecting the operation of other GDZ(s) 115 in the garment.

In some embodiments, multiple GDZ(s) 115 may be configured to form a self-assembling network. A self-assembling network may, for example, automatically establish communication links between GDZ(s) 115 without manual configuration or intervention. For example, when a user 105 dons a RAP(s) 110 equipped with multiple GDZ(s) 115, the GDZ(s) 115 may automatically discover each other and establish a mesh network. The mesh network may, for example, enable GDZ(s) 115 to communicate with each other and/or with a RAD(s) 165 through multiple communication paths, thereby advantageously providing redundancy and/or increased reliability.

The self-assembling network may, for example, utilize wireless communication protocols configured for automatic device discovery and pairing. For example, the GDZ(s) 115 may utilize Bluetooth Low Energy (BLE) with automatic pairing capabilities. In some implementations, the GDZ(s) 115 may utilize mesh networking protocols (e.g., Bluetooth Mesh, Zigbee, Thread) that enable devices to relay messages through intermediate nodes. Such protocols may, for example, advantageously extend the effective communication range and/or increase reliability by providing multiple communication paths between a GDZ(s) 115 and the RAD(s) 165.

The self-assembling network may, for example, be configured to dynamically adapt to changes in network topology. For example, if a GDZ(s) 115 is removed from the garment, damaged, or experiences a power failure, the remaining GDZ(s) 115 may automatically reconfigure the network to maintain connectivity. The network may, for example, implement routing algorithms (e.g., shortest path, load balancing) to achieve target communication efficiency and/or power consumption. In some embodiments, the self-assembling network may include a designated coordinator node (e.g., a WARM(s) 135, a designated GDZ(s) 115, the RAD(s) 165) that manages network formation, device authentication, and/or data aggregation.

Self-assembling networks may, for example, advantageously reduce setup time and/or user burden by eliminating manual pairing and configuration steps. The automatic network formation may, for example, occur transparently to the user 105, such as during a configuration method (e.g., as disclosed at least with reference to FIG. 3). The self-assembling network may, for example, advantageously increase system scalability, such as by enabling easy addition of new GDZ(s) 115 to the garment without reconfiguration of existing GDZ(s) 115. For example, various embodiments such as disclosed in this context and/or elsewhere herein may be ad-hoc may advantageously enable ad-hoc instrumentation (e.g., couple GDZs onto existing garments).

In some embodiments, GDZ(s) 115 may be configured for peer-to-peer (P2P) communication. Peer-to-peer communication may, for example, enable direct data exchange between GDZ(s) 115 without routing through a central hub (e.g., WARM(s) 135) or RAD(s) 165. For example, a first GDZ(s) 115 positioned at a front torso region may communicate directly with a second GDZ(s) 115 positioned at a rear torso region. Such communication may, for example, enable coordinated DIRE detection and/or classification based on data fusion from multiple GDZ(s) 115.

Peer-to-peer communication may, for example, enable distributed processing of sensor data. For example, multiple GDZ(s) 115 may collaborate to determine whether a detected pressure change corresponds to a DIRE or a false positive (e.g., caused by normal movement, environmental factors). A first GDZ(s) 115 detecting a pressure change may, for example, query neighboring GDZ(s) 115 for corroborating sensor data (e.g., motion sensor input, physiological sensor input). If multiple GDZ(s) 115 detect simultaneous or temporally correlated events, the intensity of a DIRE may be increased, which may be used to increase alert urgency.

In some implementations, peer-to-peer communication may enable GDZ(s) 115 to share power and/or computational resources. For example, a GDZ(s) 115 with depleted battery may enter a low-power mode and rely on neighboring GDZ(s) 115 to relay its sensor data to the RAD(s) 165. Such embodiments may, for example, advantageously extend the operational lifetime of the system by load balancing across multiple GDZ(s) 115.

Peer-to-peer communication may, for example, be implemented using direct wireless links (e.g., Bluetooth peer-to-peer, Wi-Fi Direct, ultra-wideband (UWB)). The communication protocol may, for example, include security features (e.g., encryption, authentication), such as to prevent unauthorized access and/or data tampering. In some embodiments, the GDZ(s) 115 may establish peer-to-peer connections only with other GDZ(s) 115 belonging to the same garment and/or user 105, such as determined by shared cryptographic keys and/or unique identifiers assigned during manufacturing or configuration.

In some embodiments, RAP(s) 110 worn by multiple users (e.g., multiple officers, multiple first responders) may be configured for peer-to-peer communication. For example, a first RAP(s) 110 worn by a first user 105 may communicate directly with a second RAP(s) 110 worn by a second user. Such communication may, for example, enable coordinated situational awareness and/or mutual assistance in the event of a DIRE.

Peer-to-peer communication between RAPs may, for example, be facilitated by WARM(s) 135, RAD(s) 165, and/or individual GDZ(s) 115. For example, the WARM(s) 135 of a first RAP(s) 110 may establish a wireless connection (e.g., Bluetooth, Wi-Fi, mesh network) with the WARM(s) 135 of a second RAP(s) 110. The RAPs may, for example, exchange status information, including active GDZ(s) 115, detected DIRE(s), physiological sensor input(s) 515, and/or location data (e.g., GPS coordinates). Such information exchange may, for example, advantageously enable team members to monitor each other's status in real-time.

In some implementations, peer-to-peer communication between RAPs may advantageously enable automatic alert escalation. For example, if a first user 105 experiences a DIRE and fails to respond to an alert (e.g., as determined in decision point 425 of FIG. 4), the first user's RAP(s) 110 may automatically transmit an alert to nearby RAPs worn by other users. The nearby users may, for example, receive a notification on their RAD(s) 165 indicating that a team member are to be assisted, along with the team member's location and/or injury details. Such embodiments may, for example, advantageously enable rapid peer assistance before emergency responders arrive.

Peer-to-peer communication between RAPs may, for example, utilize ad-hoc networking protocols that enable dynamic network formation without pre-existing infrastructure (e.g., cellular towers, Wi-Fi access points). For example, the RAPs may form a mobile ad-hoc network (MANET) in which each RAP acts as both a client and a router, relaying messages for other RAPs. Such networks may, for example, advantageously maintain connectivity in environments with limited or no cellular coverage (e.g., remote areas, underground facilities, disaster zones).

In some embodiments, peer-to-peer communication between RAPs may include proximity-based features. For example, RAPs within a predetermined proximity (e.g., within 10 meters, within 50 meters, within line-of-sight) may automatically establish peer-to-peer connections. The proximity determination may, for example, be based on received signal strength indication (RSSI), time-of-flight measurements, and/or GPS coordinates. Proximity-based networking may, for example, advantageously enable formation of dynamic teams (e.g., officers responding to the same incident) without manual configuration.

In some embodiments, systems may implement a hierarchical network architecture combining standalone, self-assembling, and/or peer-to-peer elements. For example, GDZ(s) 115 within a single RAP(s) 110 may form a self-assembling mesh network with peer-to-peer communication capabilities. The GDZ(s) 115 may communicate with a WARM(s) 135 and/or RAD(s) 165 that serves as a gateway to external networks (e.g., communication network(s) 170, dispatch system(s) 175). Multiple RAPs worn by different users may, for example, form a higher-level peer-to-peer network, with each RAP's WARM(s) 135 or RAD(s) 165 serving as a node in the inter-RAP network.

The hierarchical architecture may, for example, implement different communication protocols at different levels. For example, intra-RAP communication (between GDZ(s) 115 within a single garment) may utilize Bluetooth Mesh for low-power, short-range communication. Inter-RAP communication (between RAPs worn by different users) may utilize Wi-Fi Direct or cellular communication for longer range and/or higher bandwidth. Communication with external systems (e.g., dispatch system(s) 175) may utilize cellular networks, satellite communication, and/or internet connectivity.

Hybrid architectures may, for example, dynamically adapt communication strategies based on available resources and/or operational requirements. For example, in an environment with reliable cellular coverage, RAPs may communicate with dispatch systems via cellular networks. If cellular coverage is lost, the RAPs may automatically switch to peer-to-peer mesh networking to maintain connectivity with each other and/or attempt to relay messages through RAPs that still have cellular connectivity. Such adaptive behavior may, for example, advantageously provide robust communication in diverse and/or changing operational environments.

In some embodiments, peer-to-peer and self-assembling networks may implement security measures to prevent unauthorized access and/or ensure data integrity. For example, GDZ(s) 115 and/or RAPs may utilize cryptographic authentication protocols to verify the identity of peer devices before establishing communication links. Authentication may, for example, be based on pre-shared keys, public key infrastructure (PKI), and/or certificate-based authentication.

Data transmitted between GDZ(s) 115 and/or RAPs may, for example, be encrypted, such as to prevent eavesdropping and/or tampering. Encryption may, for example, utilize symmetric encryption algorithms (e.g., AES), such as for efficiency or asymmetric encryption algorithms (e.g., RSA, elliptic curve cryptography) for key exchange. In some implementations, the system may implement end-to-end encryption. Encryption may, for example, facilitate data remaining encrypted from the originating GDZ(s) 115 to the destination (e.g., RAD(s) 165, dispatch system(s) 175).

The system may, for example, implement access control mechanisms to restrict which devices can join the network and/or access sensitive data. For example, only GDZ(s) 115 and RAPs belonging to authorized users (e.g., verified law enforcement officers, registered first responders) may be permitted to join the peer-to-peer network. Access control may, for example, be enforced through device whitelisting, role-based access control, and/or multi-factor authentication.

In some embodiments, the system may include network management capabilities for monitoring and/or optimizing the performance of self-assembling and peer-to-peer networks. For example, a RAD(s) 165 or dedicated network management device may collect network performance metrics, including communication latency, packet loss rate, battery levels of GDZ(s) 115, and/or network topology. The network management system may, for example, identify performance bottlenecks, failing devices, and/or security threats.

Network management may, for example, advantageously enable remote configuration and/or firmware updates for GDZ(s) 115 and/or RAPs. For example, a system administrator may remotely update DIRE detection parameters, communication protocols, and/or security credentials across multiple devices in parallel. Such capabilities may, for example, advantageously enable rapid deployment of updates and/or security patches without physical access to each device.

In some implementations, the system may implement self-healing capabilities. For example, if a GDZ(s) 115 or communication link fails, the network may automatically reconfigure to route data through alternative paths. The system may, for example, generate alerts to notify users and/or administrators of device failures, which may advantageously enable timely maintenance and/or replacement. Self-healing networks may, for example, advantageously increase system reliability and/or reduce downtime.

In some embodiments, peer-to-peer and self-assembling network architectures may be designed for scalability to support large numbers of users and/or devices. For example, the system may support networks comprising tens, hundreds, or thousands of RAPs and/or GDZ(s) 115. Scalability may, for example, be achieved through hierarchical network structures, efficient routing algorithms, and/or distributed processing.

The system may, for example, be designed for interoperability with existing communication infrastructure and/or third-party devices. For example, the RAPs may be configured to communicate with existing dispatch systems (e.g., computer-aided dispatch (CAD) systems), emergency response networks (e.g., RapidSOS), and/or wearable devices (e.g., smartwatches, body cameras). Interoperability may, for example, be achieved through standardized communication protocols (e.g., Bluetooth, Wi-Fi, cellular standards) and/or application programming interfaces (APIs).

In some implementations, the system may support integration with Internet of Things (IoT) platforms and/or cloud services. For example, data from multiple RAPs may be aggregated in a cloud-based platform for analytics, reporting, and/or long-term storage. Cloud integration may, for example, enable advanced features such as machine learning-based DIRE detection, predictive maintenance, and/or fleet management for organizations deploying RAPs across large numbers of personnel.

In some embodiments, the system may include multiple independent wireless communication modules, each associated with a distinct GDZ(s) 115. For example, a first wireless communication module may be associated with a first GDZ(s) 115 positioned at a front portion of the garment, and a second wireless communication module may be associated with a second GDZ(s) 115 positioned at a rear portion of the garment. Each wireless communication module may, for example, independently establish a wireless connection (e.g., Bluetooth connection) with the RAD(s) 165. The RAD(s) 165 may, for example, be configured to distinguish signals from each wireless communication module based on unique identifiers (e.g., Bluetooth device addresses, assigned identifiers). Such embodiments may, for example, advantageously provide precise injury localization without a centralized hub. The independent wireless modules may, for example, reduce system complexity and/or manufacturing costs, such as by reducing or eliminating wired interconnections between GDZs.

In some embodiments, the system may be configured to integrate with external consumer wearable devices. The external wearable devices may include, by way of example and not limitation, smartwatches, fitness trackers, and/or other health monitoring devices (e.g., medical monitors). The wearable sensor(s) 255 may, for example, be communicably coupled to the RAD(s) 165 via wireless communication protocols (e.g., Bluetooth, Wi-Fi, proprietary protocols). The RAD(s) 165 may, for example, receive physiological sensor input(s) 515 from the external wearable device(s). The physiological sensor input(s) may include, by way of example and not limitation, heart rate data, heart rate variability data, blood oxygen saturation (SpO2) data, electrocardiogram (ECG) data, body temperature data, activity level data, step count data, calorie expenditure data, sleep quality data, and/or stress level indicators.

The DIRE detection engine(s) 225 may, for example, be configured to fuse the physiological sensor input(s) 515 from the external wearable device(s) with signals from the GDZ(s) 115. For example, the DIRE detection engine(s) 225 may correlate a detected pressure change in a GDZ(s) 115 with a sudden spike in heart rate, a drop in blood oxygen saturation, and/or an irregular heart rhythm detected by the external wearable device. Such correlation may, for example, advantageously enhance accuracy of DIRE detection, reduce false positive alerts, and/or enable classification of DIRE severity. The integration of external wearable devices may, for example, advantageously reduce or eliminate reliance on dedicated physiological sensors embedded in the garment and/or WARM(s) 135, thereby reducing hardware costs, system complexity, and/or maintenance requirements.

In some embodiments, a RAP may be configured with a simplified coverage area design. For example, the garment may include GDZ(s) 115 positioned only at a front portion and a rear portion of the torso, without extending to side regions, collarbone regions, and/or shoulder regions. Such embodiments may, for example, advantageously provide universal fit compatibility across different body sizes and/or armor carrier configurations. The simplified design may, for example, reduce manufacturing complexity and/or cost by minimizing the number of distinct GDZ shapes required. In some implementations, the front and rear GDZ(s) 115 may be sized according to standardized sizing categories (e.g., small, medium, large) to accommodate different user body dimensions while maintaining vacuum seal integrity and/or detection reliability.

In some embodiments, the system architecture may avoid a centralized wired communication hub. For example, rather than routing signals from multiple GDZ(s) 115 through a central wired hub before transmission to the RAD(s) 165, each GDZ(s) 115 may include its own wireless communication module(s) 240 configured to communicate directly with the RAD(s) 165. Such embodiments may, for example, advantageously reduce manufacturing costs by eliminating the hub hardware and associated wiring harnesses. The elimination of the centralized hub may, for example, advantageously facilitate maintaining vacuum seal integrity of the sealed volume(s) 260 by avoiding wired connections that penetrate the boundary wall(s) 150. The wireless architecture may, for example, reduce assembly complexity and/or increase reliability by reducing the number of physical connection points subject to wear and/or failure.

In some embodiments, the system may be designed with cost reduction as a design objective. For example, reduction of intermediate communication modules (e.g., centralized hubs, wired harnesses between GDZs) may advantageously reduce component costs, assembly labor costs, and/or quality control costs. The use of standardized wireless communication modules (e.g., commercially available Bluetooth modules) may, for example, leverage economies of scale and/or reduce procurement costs. The simplified GDZ design (e.g., front and rear panels) may reduce tooling costs, inventory complexity, and/or manufacturing setup time. In some implementations, the power storage module(s) 250 may be configured with long-life, low-cost batteries (e.g., coin cell batteries) that reduce replacement frequency and/or maintenance costs. Such cost reduction strategies may, for example, advantageously enable broader market adoption and/or increased profit margins.

In some embodiments, wireless communication architecture may advantageously enhance vacuum seal integrity. For example, traditional wired connections that penetrate the boundary wall(s) of a sealed volume(s) 260 may create potential leak paths that compromise the vacuum seal over time. Wireless designs may, for example, eliminate all physical penetrations of the sealed volume(s) 260. An unpenetrated boundary may, for example, advantageously provide a (e.g., robust) completely sealed enclosure. The sealed enclosure may, for example, be manufactured using continuous sealing processes (e.g., heat sealing, ultrasonic welding, adhesive bonding), such as without accommodation for wire pass-throughs.

In some implementations, the sealed enclosure may be manufactured using bag forming processes. Bag forming may, for example, include thermoforming of polymer films (e.g., polyethylene, polypropylene, polyurethane, nylon) into three-dimensional shapes. The thermoformed films may, for example, be sealed along joining seams, such as peripheral edges, to create the sealed volume(s) 260. In some embodiments, the bag forming process may include vacuum forming, wherein a heated polymer sheet is drawn over a mold using vacuum pressure to create the desired shape. The formed bag may, for example, be sealed using heat sealing. Heat sealing may include opposing surfaces of thermoplastic material being brought into contact and heated (e.g., via heated bars, impulse heating, hot air), such as to create a molecular bond. Heat sealing may, for example, advantageously provide a hermetic seal without additional adhesives or fasteners.

In some embodiments, a sealed enclosure may be manufactured using film sheet fusing techniques. Film sheet fusing may, for example, include laminating multiple layers of polymer film together to create a composite boundary wall(s) 150. The lamination process may, for example, utilize heat, pressure, solvents, and/or adhesive to fuse the layers. In some implementations, the film sheets may include barrier layers (e.g., aluminum foil, metallized polymer, ethylene vinyl alcohol (EVOH)). Barrier layers may, for example, reduce gas permeability and/or enhance vacuum retention. The fused film sheets may, for example, be cut to desired shapes and sealed along edges, such as, for example, using continuous sealing equipment (e.g., band sealers, rotary sealers). Film sheet fusing may, for example, advantageously enable customization of barrier properties, mechanical strength, and/or flexibility of the boundary wall(s) 150.

In some embodiments, adhesive bonding may be utilized to create the sealed enclosure. Adhesive bonding may, for example, include applying pressure-sensitive adhesives (PSAs), hot-melt adhesives, and/or reactive adhesives (e.g., epoxy, polyurethane) to join surfaces of the boundary wall(s). The adhesive may, for example, be applied in a continuous bead along sealing surfaces and cured (e.g., via heat, UV light, moisture) to create a hermetic seal. In some implementations, double-sided adhesive tape may be used to bond overlapping portions of polymer film. Adhesive bonding may, for example, advantageously enable joining of dissimilar materials and/or provide flexibility in manufacturing processes. The adhesive may, for example, be selected based on compatibility with the boundary wall material, required bond strength, and/or resistance to environmental factors (e.g., temperature, humidity, chemical exposure).

In some embodiments, ultrasonic welding may be employed to create sealed seams. Ultrasonic welding may, for example, utilize high-frequency mechanical vibrations (e.g., 20 kHz to 40 kHz) to generate localized heating at the interface between polymer surfaces. The localized heating may, for example, cause the polymer to soften and fuse together, creating a molecular bond. Ultrasonic welding may, for example, advantageously provide rapid cycle times, precise control of weld parameters, and/or minimal thermal distortion of surrounding material. The ultrasonic welding process may, for example, be particularly suitable for thin-gauge polymer films used in vacuum-sealed GDZ(s) 115.

In some implementations, the sealed enclosure may be manufactured using radio frequency (RF) welding. RF welding may, for example, utilize electromagnetic energy in the radio frequency spectrum (e.g., 27.12 MHz) to heat and fuse thermoplastic materials containing polar molecules (e.g., polyvinyl chloride (PVC), polyurethane). The RF energy may, for example, cause molecular oscillation and generate heat throughout the thickness of the material, enabling welding of thicker materials and/or multiple layers (e.g., in a single operation). RF welding may, for example, advantageously provide uniform heating, strong hermetic seals, and/or the ability to weld complex geometries.

In some embodiments, the manufacturing process may include vacuum evacuation of the sealed volume(s) 260 after sealing. For example, a vacuum port may be temporarily incorporated into the boundary wall(s) 150 during manufacturing. After the sensor(s) 255, communication module(s) 240, and/or power storage module(s) 250 are positioned within the interior, the sealed volume(s) 260 may be evacuated through the vacuum port using a vacuum pump. The vacuum port may then be sealed (e.g., via heat sealing, adhesive plug, mechanical clamp) to maintain the sub-atmospheric pressure. In some implementations, the vacuum evacuation may occur through a small opening that is subsequently sealed using a continuous sealing process, which may, for example, advantageously avoid a separate vacuum port.

In some embodiments, the manufacturing process may include quality control inspection of the sealed enclosure. Quality control may, for example, include visual inspection for defects (e.g., wrinkles, contamination, incomplete seals). Quality control may, for example, include leak testing using methods such as pressure decay testing, helium leak detection, and/or bubble testing. Pressure decay testing may, for example, involve pressurizing the sealed volume(s) 260 and monitoring for pressure loss over time. Helium leak detection may, for example, involve introducing helium into the sealed volume(s) 260 and using a mass spectrometer to detect helium escaping through leaks. Bubble testing may, for example, involve submerging the sealed enclosure in water and observing for bubble formation indicating leaks. Quality control inspection may, for example, advantageously ensure that each GDZ(s) 115 meets vacuum seal integrity specifications before deployment.

Such embodiments may, for example, advantageously increase the longevity of the vacuum seal, reduce manufacturing defect rates, and/or increase reliability of DIRE detection over the operational lifetime of the garment. The elimination of wire pass-throughs may, for example, simplify manufacturing tooling, reduce process steps, and/or enable higher production throughput. The continuous sealing processes may, for example, be automated using robotic systems and/or continuous production lines, thereby reducing labor costs and/or enhancing manufacturing consistency.

In some embodiments, a RAP may be provided with a universal fit strategy. For example, a ‘universal fit’ configuration may advantageously accommodate various body sizes and/or armor carrier configurations. For example, the GDZ(s) 115 may be configured in (e.g., in RAP portions with) standardized sizes (e.g., small, medium, large, extra-large) based on torso dimensions. Each size may, for example, include front and rear panels dimensioned to provide adequate coverage for the corresponding body size range. In some implementations, the garment may include adjustable coupling modules (e.g., adjustable straps, elastic panels, hook-and-loop fasteners) that enable fit customization within each size category. The universal fit strategy may, for example, advantageously reduce inventory complexity, simplify procurement decisions, and/or enable rapid deployment without custom fitting. In some embodiments, the GDZ(s) 115 may be configured as insertable panels that can be positioned within pockets of existing armor carriers. Such embodiments may, for example, advantageously provide retrofit compatibility with legacy equipment.

In some embodiments, the RAD(s) 165 may be configured to receive signals from multiple GDZ(s) 115 (e.g., in parallel). The RAD(s) 165 may, for example, perform injury localization based on which GDZ(s) 115 report pressure changes. For example, if a first GDZ(s) 115 positioned at the front torso reports a pressure change while a second GDZ(s) 115 positioned at the rear torso does not, the DIRE detection engine(s) 225 may determine that the injury is localized to the front torso region. The DIRE package(s) 530 may, for example, include injury location data specifying the affected body region. The alert displayed on the RAD(s) 165 (e.g., as shown in FIG. 14) may, for example, visually indicate the affected GDZ(s) 115 (e.g., via the GDZ display 1440 and active GDZs 1445). In some implementations, the system may be configured to detect multiple simultaneous impacts by identifying multiple GDZ(s) 115 reporting pressure changes within a predetermined time window (e.g., within 1 second, within 5 seconds). Such embodiments may, for example, advantageously provide detailed injury assessment information to emergency responders and/or medical personnel.

In some embodiments, the GDZ(s) 115 may be configured as modular, replaceable units. For example, each GDZ(s) 115 may be insertable into a corresponding pocket or cavity within the garment. After a DIRE event that compromises the vacuum seal of a GDZ(s) 115, the affected GDZ(s) 115 may be removed and replaced with a new GDZ(s) 115 without replacement of the entire garment. The modular design may, for example, advantageously reduce lifecycle costs and/or enable rapid return to service. In some implementations, the GDZ(s) 115 may include visual indicators (e.g., color-changing materials, indicator windows) that provide visual confirmation of vacuum seal integrity, thereby facilitating inspection and/or maintenance. The wireless communication architecture may, for example, advantageously support modular replacement by enabling automatic pairing and/or configuration of replacement GDZ(s) 115 with the RAD(s) 165 without manual reconfiguration.

Some embodiments may encompass various components, display technologies, chip technologies, interface technologies, power supply technologies, power storage technologies, server architectures, personal device architectures, portable personal computing devices, and/or software architectures.

For example, processor(s) may include central processing units (CPUs). CPUs may, for example, serve as the ‘brain’ of computer systems, such as by executing instructions and/or processing data, for example. A processor may, for example, include an arithmetic logic unit (ALU), a control unit, and/or numerous registers. Processor(s) may, for example, include graphics processing units (GPUs). GPUs may, for example, be configured to render images, videos, and/or animations. GPUs may, for example, advantageously provide greater speed for parallel processing tasks. Accordingly, GPUs may, for example, be advantageously used for tasks with intensive graphical computations.

Some embodiments may, for example, include application-specific integrated circuits (ASICs). ASICs may, for example, be custom-designed circuits (e.g., chips) tailored for specific applications. ASICs may, for example, provide high performance and efficiency.

Some embodiments may, for example, include field-programmable gate arrays (FPGAs). FPGAs may be configured, for example, as reconfigurable chips that can be programmed to perform various functions. FPGAs may, for example, advantageously be used in prototyping and/or specialized computing tasks.

Microprocessors may, for example, be configured as general-purpose chips. Microprocessors may, for example, execute instructions from software applications. As such, microprocessors may advantageously be utilized, for example, in a wide range of devices, from desktop computers to embedded systems.

Memory modules, may, for example, include volatile memory (RAM) and/or non-volatile memory (ROM). RAM may, for example, be used for temporary data storage. ROM may, for example, store firmware and/or system-level software.

Storage devices may include, for example, hard disk drives (HDDs), solid-state drives (SSDs), and/or optical drives. Storage devices may, by way of example and not limitation, store a device operating system(s), applications, and/or user data.

Input/output (I/O) interfaces may include, by way of example and not limitation, data ports, graphics ports, and/or audio ports. Data ports may include, for example, USB ports (e.g., USB-A, USB-C, USB-Mini, USB-Micro), Ethernet (e.g., RJ45), SATA ports, serial, and/or parallel ports. Graphics ports may include, for example, HDMI ports, VGA ports, and/or Display Port ports. Some ports may, for example, be multi-purpose (e.g., USB-C may carry audio, graphics, and/or other data). Audio ports may include, for example, audio jacks. I/O interfaces may, for example, facilitate communication between the computer and peripheral devices.

Display technologies may include, by way of example and not limitation, liquid crystal displays (LCDs). LCDs may, for example, be used in monitors, laptops, and/or televisions. LCDs may, for example, modulate light passing through liquid crystals to produce images. Display technologies may include, for example, light emitting diode (LED) displays. LED displays may, for example, utilize an array of LEDs for back lighting and/or as a main display technology. LED displays may, for example, offer enhanced brightness and/or color accuracy (e.g., compared to LCDs). Organic light emitting diode (OLED) displays, for example, may employ organic compounds that emit light when an electric current is applied. OLED displays may, for example, advantageously provide high contrast ratios and/or fast response times. Display technologies may, for example, include electronic paper displays (EPDs), which may also be known as e-ink displays. EPDs may, for example, be employed in e-readers. EPDs may, for example, deliver a paper-like reading experience, reduce eye strain, and/or reduce power consumption.

Interface technologies may, for example, encompass various devices that enable user interaction with a computer system. Some embodiments may, for example, include a mouse(s). A mouse may, for example, be configured as a pointing device that detects motion and translates it into cursor movement on the screen. As such, a mouse may, for example, advantageously allow a user to interact with graphical user interfaces.

Keyboards may, for example, be configured for text entry and/or command execution. A keyboard may, for example, include multiple keys (e.g., arranged in a standard layout).

Touch inputs may, for example, enable direct interaction with a display or other input device through gestures such as tapping, swiping, and pinching.

Some embodiments may, for example, include an audio capture device(s), such as a microphone(s). For example, a device may be configured to record voice inputs. Some embodiments may, for example, leverage voice recognition technology, allowing users to control devices and/or enter text using spoken commands.

Additional interface technologies which may be included in some embodiments may, by way of example and not limitation, include track pads, joysticks, styluses, and/or game controllers.

Various embodiments may include one or more power supply and/or storage technologies. For example, power supplies may convert electrical power from an outlet into usable power for a device's components. A power supply may, for example, include one or more transformers, rectifiers, and/or regulators. Batteries may, for example, advantageously provide portable power for devices such as laptops, smartphones, and tablets. Batteries of one or more chemistries may be used, including, by way of example and not limitation, lithium-ion, and/or nickel-metal hydride.

In some embodiments, devices disclosed herein may be configured as and/or connected in a server architecture. A server architecture may, for example, be configured to advantageously provide scalable computing resources, such as in enterprise environments, for example. Some embodiments may, for example, include blade servers. Blade servers may, for example, be configured as modular servers that fit into a chassis, which may advantageously allow for high-density computing and/or increase space and/or power efficiency in data centers. Rack servers may, for example, be configured to be mounted in standardized racks. Rack servers may, for example, advantageously provide scalable computing resources. Cloud servers may, for example, include virtualized servers, which may be hosted in data centers. Cloud servers may, for example, advantageously offer flexible and/or scalable resources to users over the internet.

In some embodiments, devices disclosed herein may be configured as and/or connected to a personal device architecture, for example. Personal device architectures may include, for example, desktop computers. A desktop computer may, for example, include a tower, monitor, keyboard, and mouse, and may be used, for example, for a wide range of applications, from office work to gaming. Laptops may, for example, be configured as portable computers. The portable computers may, for example, integrate a display, keyboard, and position input (e.g., track pad) into a single unit. Laptops may, for example, be used for mobile computing and may, for example, perform many of the same tasks as desktops. Portable personal computing devices may, by way of example and not limitation, include smartphones. Smartphones may be configured, for example, as compact devices that combine computing capabilities with telecommunication functions. These devices may include, by way of example and not limitation, touchscreens, cameras, and/or various sensors. Portable personal computing devices may include, for example, smartwatches. Smartwatches may, for example, be configured as wearable devices. Smartwatches may, for example, provide notifications, fitness tracking, and/or other functionalities. Smartwatches may, for example, be configured to pair with smartphones and/or other computer(s) for extended capabilities. Portable personal computing devices may, for example, include tablets. Tablets may, for example, be configured as portable devices with touchscreens larger than smartphones. Tablets may, for example, advantageously be used for tasks such as web browsing, media consumption, and/or productivity applications.

Engines and/or modules disclosed herein may be configured in one or more software architectures. Software architectures may, for example, include operating systems. Operating systems may, for example, manage hardware resources and/or provide a platform for running applications.

Software architectures may, for example, include application software. Application software may include, for example, programs designed for specific tasks.

Software architectures may, for example, include middleware. Middleware may, for example, be configured to provide services to software applications beyond those offered by the operating system. Middleware may, for example, include components such as web servers, database management systems, and/or message brokers.

Software architectures may include, for example, firmware. Firmware may, for example, be configured as low-level software embedded in hardware devices. Firmware may, for example, control functions of the hardware devices. Firmware may, by way of example and not limitation, be stored in ROM and/or flash memory.

Software architectures may, for example, include virtualization technology. Virtualization technology may, for example, be configured to allow multiple virtual machines to run on a single physical machine. Virtualization technology may, for example, advantageously enable efficient resource utilization and/or isolation.

Various embodiments may, for example, include connection and/or communication technologies. Such technologies may, by way of example and not limitation, be configured to facilitate the exchange of data across various distances and/or environments. Long-range communication technologies may, by way of example and no t limitation, include cellular networks, satellite communications, and/or broadband internet connections. Cellular networks, such as 4G LTE and 5G, may advantageously provide wireless connectivity over large areas. Cellular networks may, for example, enable devices (e.g., mobile devices) to access the internet, make calls, and/or otherwise transmit data. Satellite communications may, for example, advantageously provide global coverage, which may be particularly useful in remote and/or underserved regions where terrestrial infrastructure is limited. Broadband internet connections may include, by way of example and not limitation, fiber-optic, DSL, and/or cable. Broadband may, for example, advantageously provide high-speed internet access to devices such as for activities including streaming, online gaming, and/or remote work.

Local communication technologies may, for example, encompass methods for connecting devices within a limited area, such as a home, office, and/or campus. Local communication technologies include, for example, Wi-Fi. Wi-Fi may, for example, connect device(s) to a wireless local area network (WLAN) and/or access the internet and/or share resources (e.g., printers, storage). Wired communication technology, such as Ethernet, may, for example, advantageously provide reliable and/or high-speed connections between devices in a local network, such as, by way of example and not limitation, desktops, servers, and/or network switches. Power-line communication (PLC) may, for example, enable data transmission over existing electrical wiring. PLC may, for example, advantageously provide an option for connecting devices in locations where Wi-Fi signals may be weak or unreliable.

Near field communication (NFC) and BLUETOOTH are examples of short-range communication technologies. Short-range communication technologies may, by way of example and not limitation, be configured to connect devices within a few centimeters to several meters. NFC may, for example, include wireless technology configured to enable contactless communication between devices. NFC may, for example, be configured for use with mobile payments, access control, and/or data transfer. Its short range may, for example, advantageously enhance security by relying on close proximity for communication. Bluetooth may, for example, provide wireless connectivity over a range of meters (e.g., up to 100 meters). Bluetooth may, for example, advantageously be deployed in implementations connecting peripherals such as keyboards, mice, headphones, and/or wearable devices, and/or for transferring files between devices.

As an illustrative example, an injury threat event detection apparatus may include a garment. The garment may include at least one garment detection zone (GDZ). Each GDZ may include a vacuum-sealed interior region. At least one sensor module may be disposed within the interior region of each GDZ. The sensor module may be configured to detect a change in pressure within the interior region. A remote alert device (RAD) may be communicably coupled to the sensor module of each GDZ. The RAD may be configured to receive a signal from the sensor module indicating the detected change in pressure. The RAD may detect an injury threat event based on the signal. The RAD may trigger an alert in response to detecting the injury threat event.

The change in pressure may be an elevation of pressure from sub-atmospheric pressure to atmospheric pressure. Detecting the injury threat event may include detecting a lack of sub-atmospheric pressure in one or more of the at least one GDZ.

The RAD may be configured to display the alert to a wearer of the garment.

The alert may include information identifying a location of the injury threat event on the garment. The location may be determined at least based on a predetermined position relative to the garment of the GDZ in which the change in pressure is detected.

The alert may include information identifying a type of the detected injury threat event.

The sensor module may include expandable media and an electric attribute detection module. The expandable media may be configured to expand upon pressure change. The expansion may alter an electric attribute detected by the electric attribute detection module.

The garment may include at least one physiological sensor. The physiological sensor may be configured to generate physiological sensor input.

The at least one GDZ may include a continuous sealed boundary. The continuous sealed boundary may be free of communication conduits traversing the continuous sealed boundary. The sensor module may be disposed within the continuous sealed boundary.

The RAD may be communicably coupled to the at least one GDZ via a wearable automatic response module (WARM). The WARM may be in wireless communication with the RAD. When the WARM is releasably coupled to the garment, the WARM may be in wired communication with the at least one GDZ.

The garment may be configured as a vest.

The garment may include at least one coupling module. The coupling module may be configured to receive the at least one GDZ.

As an illustrative example, a method of detecting an injury threat event may include providing a detection apparatus. The detection apparatus may include a responsive apportioned garment. The garment may include at least one garment detection zone (GDZ). Each GDZ may include a vacuum-sealed interior region. At least one sensor module may be disposed within the interior region of each GDZ. The sensor module may be configured to detect a change in pressure within the vacuum-sealed interior region. The method may include communicably coupling a remote alert device (RAD) to the sensor module of each GDZ. The method may include receiving a signal from the sensor module indicating the change in pressure detected. The method may include detecting an injury threat event based on the signal. The method may include triggering an alert in response to detecting the injury threat event.

As an illustrative example, an injury threat event detection apparatus may include a responsive apportioned garment. The garment may include at least one garment detection zone (GDZ). Each GDZ may include a region of sub-atmospheric pressure. The apparatus may include means for detecting a loss of the sub-atmospheric pressure. The apparatus may include means for generating an alert in response to detecting the loss of sub-atmospheric pressure.

It will be understood that various modifications can be made within the scope of this disclosure. For example, one or more advantageously results may be achieved if components are removed, added, multiplied, scaled, and/or rearranged, and/or if steps in a method are omitted, added, repeated, and/or performed in a different order. Therefore, other implementations are contemplated within the scope of the following claims.

1. An apparatus suitable for injury threat event detection, the apparatus comprising:
a garment comprising at least one garment detection zone (GDZ), wherein each GDZ comprises an interior region that is vacuum-sealed;
at least one sensor module disposed within the interior region of each GDZ, the at least one sensor module configured to detect a change in pressure within the interior region; and
a remote alert device (RAD) communicably coupled to the at least one sensor module of each GDZ, the RAD configured to:
receive a signal from the at least one sensor module indicating the change in pressure detected,
detect an injury threat event based on the signal, and
trigger an alert in response to detecting the injury threat event.

2. The apparatus of claim 1, wherein the change in pressure is an elevation of pressure from sub-atmospheric pressure to atmospheric pressure, and detecting the injury threat event comprises detecting a lack of sub-atmospheric pressure in one or more of the at least one GDZ.

3. The apparatus of claim 1, wherein the RAD is configured to display the alert to a wearer of the garment.

4. The apparatus of claim 3, wherein the RAD is communicably coupled to a communication network, and the RAD is configured to transmit the alert to a dispatch system via the communication network.

5. The apparatus of claim 4, wherein the RAD is configured to transmit the alert to the dispatch system after communicating the alert to the wearer and failing to receive an alert dismissal from the wearer.

6. The apparatus of claim 1, wherein the alert includes information identifying a location of the injury threat event on the garment, determined at least based on a predetermined position relative to the garment of the at least one GDZ in which the change in pressure is detected.

7. The apparatus of claim 1, wherein the alert includes information identifying a type of the injury threat event detected.

8. The apparatus of claim 1, wherein the at least one sensor module comprises a pressure sensor configured to measure pressure within the interior region.

9. The apparatus of claim 1, wherein the at least one sensor module comprises expandable media and an electric attribute detection module, wherein the expandable media is configured to expand upon pressure change and alter an electric attribute detected by the electric attribute detection module.

10. The apparatus of claim 1, wherein the at least one sensor module comprises a piezoelectric film disposed along a surface of a boundary wall of the interior region, wherein the piezoelectric film is configured to generate the signal corresponding to the change in pressure within the interior region.

11. The apparatus of claim 1, wherein the garment further comprises at least one physiological sensor configured to generate physiological sensor input.

12. The apparatus of claim 11, wherein the at least one physiological sensor comprises at least one of a photoplethysmogram (PPG) sensor or a temperature sensor.

13. The apparatus of claim 1, wherein the at least one GDZ comprises a continuous sealed boundary free of communication conduits traversing the continuous sealed boundary, and the at least one sensor module is disposed within the continuous sealed boundary.

14. The apparatus of claim 13, wherein the at least one sensor module is wirelessly communicably coupled to the RAD.

15. The apparatus of claim 1, wherein the RAD comprises a smartphone.

16. The apparatus of claim 1, wherein the RAD is communicably coupled to the at least one GDZ via a wearable automatic response module (WARM).

17. The apparatus of claim 16, wherein the WARM is releasably coupled to the garment.

18. The apparatus of claim 17, wherein:
the WARM is in wireless communication with the RAD, and
when the WARM is releasably coupled to the garment, the WARM is in wired communication with the at least one GDZ.

19. The apparatus of claim 1, wherein the garment comprises a vest.

20. The apparatus of claim 1, wherein the garment comprises at least one coupling module configured to receive the at least one GDZ.

21. A method of detecting an injury threat event, the method comprising:
provide a detection apparatus comprising:
a responsive apportioned garment comprising at least one garment detection zone (GDZ), wherein each GDZ comprises an interior region that is vacuum-sealed;
at least one sensor module disposed within the interior region of each GDZ, the
at least one sensor module configured to detect a change in pressure within the vacuum-sealed interior region; and
communicable couple a remote alert device (RAD) to the at least one sensor module of each GDZ;
receive a signal from the at least one sensor module indicating the change in pressure detected,
detect an injury threat event based on the signal, and
trigger an alert in response to detecting the injury threat event.

22. An apparatus suitable for injury threat event detection, the apparatus comprising:
a responsive apportioned garment comprising at least one garment detection zone (GDZ), wherein each GDZ comprises a region of sub-atmospheric pressure;
means for detecting a loss of the sub-atmospheric pressure; and
means for generating an alert in response to detecting the loss of sub-atmospheric pressure.
